(19) United States
(12) Patent Application Publication
(10) Pub. No.: US 2009/0162286 A1
(43) Pub. Date: Jun. 25, 2009

(54) Phosphodiesterase 10 Inhibition as Treatment for Obesity-Related and Metabolic Syndrome-Related Conditions

(75) Inventors: Shawn C. Black, Ivoryton, CT (US); Earl M. Gibbs, Oakdale, CT (US)

Correspondence Address: PFIZER INC., PATENT DEPARTMENT, Bld 114 M/S 114, EASTERN POINT ROAD, GROTON, CT, 06340, US

(73) Assignee: Pfizer Inc.

(21) Appl. No.: 11/628,924

(22) PCT Filed: May 30, 2005
(86) PCT No.: PCT/IB05/01755

§ 371 Date: Aug. 31, 2007

Related U.S. Application Data
(60) Provisional application No. 60/577,535, filed on Jun. 7, 2004.

(51) Int. Cl. A61B 10/00; A61K 31/454; A61K 31/337; A61K 31/135; A61K 31/4985; A61K 38/10; A61K 38/17; A61P 3/04; A61P 3/10; A61P 3/00

(52) U.S. Cl. 424/9.2; 514/326; 514/449; 514/646; 514/250; 514/13; 514/12

(57) ABSTRACT

The present invention provides methods to decrease body weight and/or body fat in animals, e.g., in the treatment of overweight or obese patients (e.g., humans or companion animals), or as a means to produce leaner meat in food stock animals (e.g., cattle, chickens, pigs), methods to treat non-insulin dependent diabetes (NIDDM), metabolic syndrome, or glucose intolerance, in patients in need thereof by administering a PDE10 inhibitor (alone or in combination with another therapeutic agent), kits for the above-identified therapeutic uses, and methods of identifying PDE10 inhibitors for the above-described therapeutic uses.

### FIELD OF THE INVENTION

The present invention provides methods to decrease body weight and/or body fat in the treatment, for example, of overweight or obese patients, and methods for treating metabolic syndrome, non-insulin dependent diabetes, or glucose intolerance, by administering a phosphodiesterase 10 (PDE10) inhibitor.

### BACKGROUND

Individuals diagnosed as obese or overweight suffer increased risk for developing other health conditions such as coronary heart disease, stroke, hypertension, type 2 diabetes mellitus, dyslipidemia, sleep apnea, osteoarthritis, gall bladder disease, depression, and certain forms of cancer (e.g., endometrial, breast, prostate, and colon). The negative health consequences of obesity make it the second leading cause of preventable death in the United States and a major public health concern that imparts a significant economic and psychosocial effect on society (see, e.g., McGinnis and Foege, JAMA 270: 2207-2212, 1993).

Obesity is now recognized as a chronic disease that requires treatment to reduce its associated health risks. Although weight loss itself is an important treatment outcome, one of the main goals of obesity management is to improve cardiovascular and metabolic values to reduce obesity-related morbidity and mortality. It has been shown that 5-10% loss of body weight can substantially improve metabolic and cardiovascular values, such as blood glucose, blood pressure, and lipid concentrations. Hence, it is believed that a 5-10% reduction in body weight may reduce morbidity and mortality.

Cyclic nucleotide phosphodiesterases (PDEs) catalyze the hydrolysis of cyclic nucleotides, such as the second messengers CAMP (cyclic adenosine 3′5′-monophosphate) and cGMP (cyclic guanine 3′5′-monophosphate). Thus, PDEs play a pivotal regulator role in a wide variety of signal transduction pathways (Beavo, Physiol. Rev. 75: 725-748, 1995). For example, PDEs mediate processes involved in vision (McLaughlin et al., Nat. Genet. 4: 130-134, 1993), olfaction (Yan et al., Proc. Natl. Acad. Sci. USA 92: 9677-81, 1995), platelet aggregation (Dickinson et al. Biochem. J. 323: 371-377, 1997), aldosterone synthesis (MacFarland et al., J. Biol. Chem. 266: 136-142, 1991), insulin secretion (Zhao et al., J. Clin. Invest. 102: 869-873, 1998), T cell activation (Li et al., Science 283: 848-51, 1999), and smooth muscle relaxation (Boolell et al., Int. J. Impot. Res. 8: 47-52, 1996; Ballard et al., J. Urol. 159: 2164-171, 1998).

PDEs form a superfamily of enzymes that are subdivided into 11 major gene families (Beavo, Physiol. Rev. 75: 725-748, 1995; Beavo et al., Mol. Pharmacol. 46: 399-405, 1994; Soderling et al., Proc. Natl. Acad. Sci. USA 95: 8991-8996, 1998; Fisher et al., Biochem. Biophys. Res. Commun. 246: 570-577, 1998; Hayashi et al., Biochem. Biophys. Res. Commun. 250: 751-756, 1998; Soderling et al., J. Biol. Chem. 273: 15553-58, 1998; Fisher et al., J. Biol. Chem. 273: 15559-15564, 1998; Soderling et al., Proc. Nat. Acad. Sci. USA 96: 7071-7076, 1999; and Fawcett et al., Proc. Natl. Acad. Sci. USA 97: 3702-3707, 2000).

Each PDE gene family encodes a phosphodiesterase distinguished functionally by unique enzymatic characteristics and pharmacological profiles. In addition, each family exhibits distinct tissue, cellular, and subcellular expression patterns (Beavo et al., Mol. Pharmacol. 46: 399-405, 1994; Soderling et al., Proc. Natl. Acad. Sci. USA 95: 8991-8996, 1998; Fisher et al., Biochem. Biophys. Res. Commun. 246: 570-577, 1998; Hayashi et al., Biochem. Biophys. Res. Commun. 250: 751-756, 1998; Soderling et al., J. Biol. Chem. 273: 15553-15558, 1998; Fisher et al., J. Biol. Chem. 273: 15559-15564, 1998; Soderling et al., Proc. Natl. Acad. Sci. USA 96: 7071-7076, 1999; Fawcett et al., Proc. Natl. Acad. Sci. USA 97: 3702-3707, 2000; Boolell et al., Int. J. Impot. Res. 8: 47-52, 1996; Ballard et al., J. Urol. 159: 2164-2171, 1998; Houslay, Semin. Cell Dev. Biol. 9: 161-167, 1998; and Torphy et al., Pulm. Pharmacol. Ther. 12: 131-135, 1999). Therefore, by administering a compound that selectively regulates the activity of one family or subfamily of PDE enzymes, it is possible to regulate cAMP and/or cGMP signal transduction pathways in a cell- or tissue-specific manner.

PDE10 is identified as a unique PDE based on amino acid sequence information and distinct enzymatic activity. Homology screening of EST databases revealed PDE10, sometimes referred to as PDE10A, as the first, and so far only, member of its PDE10 gene family of phosphodiesterases (Fujishige et al., J. Biol. Chem. 274: 18438-18445, 1999; Loughney et al., Gene 234:109-117, 1999). The human, rat, and murine homologues have been cloned and N-terminal splice variants have been identified for both the rat and human genes (Kotera et al., Biochem. Biophys. Res. Comm. 261: 551-557, 1999; Fujishige et al., Eur. J. Biochem. 266: 1118-1127, 1999; Soderling et al., Proc. Natl. Acad. Sci. USA 96: 7071-7076, 1999, and Lanfear and Robas, U.S. Pat. Appl. Publ. No. 2004/0018542); there is a high degree of homology across species. PDE10 hydrolyzes cAMP and cGMP to AMP and GMP, respectively.

Current data on PDE10 expression indicates that PDE10 is uniquely localized in mammals relative to other PDE families. Messenger RNA for PDE10 is highly expressed in testis and brain (Lanfear and Robas, U.S. Pat. Appl. Publ. No. 2004/0018542; Fujishige et al., Eur. J. Biochem. 266: 1118-1127, 1999; Soderling et al., Proc. Natl. Acad. Sci. USA 96: 7071-7076, 1999; Loughney et al., Gene 234:109-117, 1999). Autoradiographs of the PDE10 antisense-labeled mouse brain sections display a highly specific hybridization signal. Dense labeling is found, so far, in three areas; dorsal striatum (caudate and putamen), ventral striatum (nucleus accumbens), and olfactory tubercle. Within the striatum and nucleus accumbens, PDE10 mRNA is highly expressed in the striatal medium spiny neurons, which represent about 95% of all neurons found in these structures. A lower density of labeling is noted in other areas, including dentate gyrus, CA layers of hippocampus, and in the granule cell layer of cerebellum.

There is very good correspondence between PDE10 mRNA localization areas and those areas classically associated with high dopamine receptor expression. In emulsion autoradiographs, dense incorporation of silver grains is found throughout the striatum, nucleus accumbens, and olfactory tubercle, and is noted to overlay the vast majority of the neuronal cell bodies in these three areas. In addition, areas which express low but measurable levels of dopamine receptors also demonstrate grain deposition, in rough correspondence with their relative DA receptor density. These include, notably, the medial and sulcal prefrontal cortices as well as dentate gyrus and the CA layers of hippocampus (Seeger et al., Brain Res. 985: 113-126, 2003).

Consistent with high mRNA levels, a high level of PDE10 protein is demonstrated in the striatum (caudate and putamen), nucleus accumbens, and olfactory tubercle. PDE10 protein is observed in the neuronal cell bodies and throughout the neuropil. Furthermore, a high level of PDE10 protein, but not PDE10 mRNA, is observed in the brain regions to which the striatal medium spiny neurons project, including the internal capsule, globus pallidus, entopeduncular nucleus, and the substantia nigra. This high level of PDE10 protein could arise from the axons and terminals of the striatal medium spiny neurons (Seeger et al., Brain Res. 985: 113-126, 2003).

### SUMMARY OF THE INVENTION

The present invention provides methods to decrease body weight and/or body fat, and methods for treating metabolic syndrome, non-insulin dependent diabetes NIDDM), or glucose intolerance, by administering a PDE10 inhibitor (alone or in combination with another therapeutic agent), as well as related kits, and methods of screening for PDE10 inhibitors for the above-described therapeutic uses.

In one preferred embodiment, the invention provides a method of treating a subject to reduce body fat or body weight, or to treat NIDDM, metabolic syndrome, or glucose intolerance, comprising administering to a subject in need thereof a therapeutically effective amount of a phosphodiesterase 10 (PDE10) antagonist. In preferred embodiments, the subject is human, the subject is overweight or obese, the PDE10 antagonist is a PDE10 selective antagonist, e.g., papaverine or 6,7-dimethoxy-4-[8-(morpholine-4-sulfonyl)-3,4-dihydro-1H-isoquinolin-2-yl]-quinazoline, and/or the antagonist is administered orally. In another preferred embodiment, the method further comprising administering a second therapeutic agent to the subject, preferably an anti-obesity agent, e.g., rimonabant, orlistat, sibutramine, bromocriptine, ephedrine, leptin, pseudoephedrine, or peptide YY₃₋₃₆, or analogs thereof.

A second aspect of the invention is a method for identifying an agent that can be used to reduce body fat or body weight, or to treat NIDDM, metabolic syndrome, or glucose intolerance, comprising (i) administering a candidate PDE10 antagonist to a test subject, and (ii) determining whether the PDE10 antagonist is effective in reducing body fat or body weight, or in treating NIDDM, metabolic syndrome, or glucose intolerance, in the test subject. As a related aspect, the method can further comprise testing the candidate PDE10 antagonist in an in vitro test for PDE10 antagonist activity prior to administering the candidate PDE10 antagonist to the test subject. In other preferred embodiments, the test subject is a laboratory animal.

Also featured as an aspect of the invention is a kit comprising a PDE10 antagonist and instructions for administering the antagonist to a subject to reduce body fat or body weight, or to treat NIDDM, metabolic syndrome, or glucose intolerance, in the subject. Preferably, the PDE10 antagonist is a PDE10 selective antagonist, e.g., papaverine or 6,7-dimethoxy-4-[8-(morpholine-4-sulfonyl)-3,4-dihydro-1H-isoquinolin-2-yl]-quinazoline. In other preferred embodiments, the kit can further comprise a second therapeutic agent, more preferably, an anti-obesity agent, e.g., rimonabant, orlistat, sibutramine, bromocriptine, ephedrine, leptin, pseudoephedrine, or peptide YY₃₋₃₆, or analogs thereof.

Those skilled in the art will fully understand the terms used herein in the description and the appendant claims to describe the present invention. Nonetheless, unless otherwise provided herein, the following terms are as described immediately below.

### DEFINITIONS

By “PDE10 inhibitor” or “PDE10 antagonist” is meant an agent that reduces or attenuates the biological activity of the PDE10 polypeptide in a cell. Such agents may include proteins, such as anti-PDE10 antibodies, nucleic acids, e.g., PDE10 antisense or RNA interference (RNAi) nucleic acids, amino acids, peptides, carbohydrates, small molecules (organic or inorganic), or any other compound or composition which decreases the activity of a PDE10 polypeptide either by effectively reducing the amount of PDE10 present in a cell, or by decreasing the enzymatic activity of the PDE10 polypeptide. Compounds that are PDE10 inhibitors include all solvates, hydrates, pharmaceutically acceptable salts, tautomers, stereoisomers, and prodrugs of the compounds. Preferably, a small molecule PDE10 inhibitor used in the present invention has an IC₅₀ of less than 10 μM, more preferably, less than 1 μM, and, even more preferably, less than 0.1 μM. An antisense oligonucleotide directed to the PDE10 gene or mRNA to inhibit its expression is made according to standard techniques (see, e.g., Agrawal et al. Methods in Molecular Biology Protocols for Oligonucleotides and Analogs, Vol. 20, 1993). Similarly, an RNA interference molecule that functions to reduce the production of PDE10 enzyme in a cell can be produced according to standard techniques known to those skilled in the art (see, e.g., Hannon, Nature 418: 244-251, 2002; Shi, Trends in Genetics 19: 9-12, 2003; Shuey et al., Drug Discovery Today 7: 1040-1046, 2002). Examples of PDE10 inhibitors include papaverine, as described in U.S. Pat Appl. Publ. No. 2003/0032579, as well the compounds disclosed in U.S. Provisional Patent Appl. No. 60/466,639, filed Feb. 18, 2004, entitled “Tetrahydroisoquinolinyl Derivatives of Quinasoline and Isoquinoline,” which are also further described herein.

Any PDE10 antagonist (inhibitor) used in the present invention is preferably also selective against some or all other PDEs, preferably, against PDE1A, PDE1B, PDE1C, PDE2, PDE3A, PDE3B, PDE4A, PDE4B, PDE4C, PDE4D, PDE5, PDE6, PDE7A, PDE7B, PDE8A, PDE8B, PDE9, and/or PDE11.

By a “selective” PDE10 inhibitor, when the agent inhibits PDE10 activity, is meant an agent that reduces PDE10 activity with an Ki at least 10-fold less, preferably, at least 100-fold less, than the Ki for inhibition of one or more other PDEs. Preferably, such agents are combined with a pharmaceutically acceptable delivery vehicle or carrier. By a “selective” PDE10 inhibitor, when the agent reduces the amount of PDE10 in a cell, is meant an agent that reduces PDE10 polypeptide in a cell, but not one or more of the other PDEs, as determined by quantitative PCR

“Decreased PDE10 activity” means a manipulated decrease in the total polypeptide activity of the PDE10 enzyme as a result of genetic disruption or manipulation of the PDE10 gene function that causes a reduction in the level of functional PDE10 polypeptide in a cell, or as the result of administration of a pharmacological agent that inhibits PDE10 activity.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “prodrug” refers to a compound that is a drug precursor which, following administration, releases the drug in vivo via a chemical or physiological process (e.g., upon being brought to physiological pH or through enzyme activity). A discussion of the synthesis and use of prodrugs is provided by Higuchi and Stella, Prodrugs as Novel Delivery Systems, vol. 14 of the ACS Symposium Series, and Bioreversible Carriers in Drug Design, ed. Edward B. Roche, American Pharmaceutical Association and Pergamon Press, 1987.

The terms “salts” and “pharmaceutically acceptable salts” refer to organic and inorganic salts of a compound, a stereoisomer of the compound, or a prodrug of the compound.

“Overweight” and the more severe “obese” conditions, in an adult person 18 years or older, constitute having greater than ideal body weight (more specifically, greater than ideal body fat) and are generally defined by body mass index (BMI), which is correlated with total body fat and the relative risk of suffering from premature death or disability due to disease as a consequence of the overweight or obese condition. The health risks increase with the increase in excessive body fat. BMI is calculated by weight in kilograms divided by height in meters squared (kg/m²) or, alternatively, by weight in pounds, multiplied by 703, divided by height in inches squared (lbs×703/in²). “Overweight” typically constitutes a BMI of between 25.0 and 29.9. “Obesity” is typically defined as a BMI of 30 or greater (see, e.g., National Heart, Lung, and Blood Institute, Clinical Guidelines on the Identification, Evaluation, and Treatment of Overweight and Obesity in Adults, The Evidence Report, Washington, D.C.: U.S. Department of Health and Human Services, NIH publication no. 98-4083, 1998). In heavily muscled individuals, the correlation between BMI, body fat, and disease risk is weaker than in other individuals. Therefore, assessment of whether such heavily muscled individuals are in fact overweight or obese may be more accurately performed by another measure such as direct measure of total body fat or waist-to-hip ratio assessment.

“Metabolic syndrome”, as defined herein, and as according to the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. no. 01-3670), occurs when a person has three or more of the following symptoms:
1. Abdominal obesity: waist circumference >102 cm in men and >88 cm in women;
2. Hypertriglyceridemia: ≧150 mg/dl (1.695 mmol/l);
3. Low HDL cholesterol: <40 mg/dl (1.036 mmol/l) in men and <50 mg/dl (1.295 mmol/l) in women;
4. High blood pressure: ≧130/85 mmHg;
5. High fasting glucose: ≧110 mg/dl (≧6.1 mmol/l); or,
   as according to World Health Organization criteria (Alberti and Zimmet, Diabet. Med. 15: 539-53, 1998), when a person has diabetes, impaired glucose tolerance, impaired fasting glucose, or insulin resistance plus two or more of the following abnormalities:
1. High blood pressure: ≧160/90 mmHg;
2. Hyperlipidemia: triglyceride concentration ≧150 mg/dl (1.695 mmol/l) and/or HDL-cholesterol <35 mg/dl (0.9 mmol/l) in men and <39 mg/dl (1.0 mmol/l) in women;
3. Central obesity: waist-to-hip ratio of >0.90 for men and >0.85 in women and/or BMI >30 kg/m²;
4. Microalbuminuria: urinary albumin excretion rate ≧20 μg/min or an albumin-to-creatinine ratio≧20 mg/kg.

By “therapeutically effective” is meant resulting in a decrease, with respect to the appropriate control, in body fat, body weight, and/or in the amelioration of one or more symptoms of NIDDM, metabolic syndrome, or glucose intolerance.

Other features and advantages of the invention will be even further apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. All publications, including published patent applications and issued patents, mentioned herein are incorporated by reference in their entireties.

### BRIEF DESCRIPTION OF THE FIGURES

FIG. 1 shows the time course of body weight changes, in grams, for the four experimental groups: wild type (WT) mice on a chow diet (WT-Chow); PDE10 knockout (KO) mice on a chow diet (KO-Chow); wild type mice on a high fat diet (WT-HFD); and PDE10 KO mice on a high fat diet (KO-HFD). WT-HFD mice exhibited an increase in body weight as compared to the WT-Chow mice. However, KO-HFD mice did not experience a similar increase as compared to KO-Chow mice.

FIG. 2 shows the same time course of body weight changes, but expresses body weight as the percentage of baseline.

FIG. 3 is a bar graph showing the body composition for the four experimental groups following the chow and high fat diets. The increased body weight in WT-HFD mice as compared to the other groups, as shown in FIG. 1 and FIG. 2, corresponded with an increase in fat mass and % body fat.

FIG. 4 is a line graph detailing the time course of food consumption, normalized for body weight, for the four experimental groups. Despite the reduced body weight gain of the KO-HFD mice as compared to WT-HFD mice, as shown in FIG. 1 and FIG. 2, KO-HFD mice consumed an equal, or slightly greater, amount of high fat diet (HFD) as compared to the WT mice.

FIG. 5 is a bar graph showing activity levels. A trend was observed towards decreased activity in the PDE10 KO mice as compared to WT mice independent of diet.

FIG. 6 depicts oxygen consumption (VO2) and shows an increase in total VO2 in the KO-HFD mice as compared to the other groups.

FIG. 7 shows that resting VO2 is also increased in the KO-HFD mice as compared to the three other experimental groups.

FIGS. 8A and 8B are line graphs showing the results of an oral glucose tolerance test in the four experimental groups using a normal chow diet (FIG. 8A) and a hi-fat diet (HFD: FIG. 8B). HFD-fed PDE10 knock out (KO) mice showed improved glucose tolerance as compared to their wild type (WT) counterparts.

FIG. 9A is a bar graph depicting the effect on food intake over time of Compound IIA in fasted CD rats using the Obesity Food Intake Model-Fasted-Induced Feeding Assay. Compound IIA was efficacious at 10 mg/kg. Rimonabant (Acomplia™) is used as a comparator at 3 mg/kg.

FIG. 9B is a bar graph depicting the effect on food intake over time of Compound IIIA in fasted CD rats using the Obesity Food Intake Model-Fasted-Induced Feeding Assay. Compound IIIA was efficacious at 10 mg/kg. Rimonabant (Acomplia™) is used as a comparator at 3 mg/kg.

FIG. 9C is a bar graph comparing the efficacy of Compound IIA and Compound IIIA in fasted CD rats using the Obesity Food intake Model-Fasted-Induced Feeding Assay at 10 mg/kg and 32 mg/kg. Rimonabant (Acomplia™) is used as a comparator at 3 mg/kg.

FIG. 10 is a line graph showing body weight changes in diet-induced obese (DIO) mice treated with Compound IVA. DIO mice were treated with Compound IVA at a dose of 15 mg/kg administered twice daily (po). Control mice were administered the vehicle, 0.5% methylcellulose.

FIG. 11 is line graph showing changes in food intake over time in diet-induced obese (DIO) mice treated with Compound IVA. DIO mice were treated with Compound IVA at a dose of 15 mg/kg administered twice daily (po). Control mice were administered the vehicle, 0.5% methylcellulose.

FIG. 12 is a line graph showing changes in oxygen consumption following treatment of diet-induced obese (DIO) mice with Compound IVA. DIO mice were treated with Compound IVA at a dose of 15 mg/kg administered twice daily (po). Control mice were administered the vehicle, 0.5% methylcellulose.

### DETAILED DESCRIPTION

The present invention is directed to methods to decrease body weight and/or body fat in an animal, e.g., in the treatment of overweight or obese patients (e.g., humans or companion animals), or as a means to produce leaner meat in food stock animals (e.g., cattle, chickens, pigs), and methods to treat metabolic syndrome, non-insulin dependent diabetes, and/or glucose intolerance in patients in need thereof by administering a PDE10 inhibitor. As disclosed in the Examples herein, PDE10 knockout mice are relatively resistant to developing increased body weight, increased adiposity, and symptoms of metabolic syndrome, subsequent to exposure to a high fat diet. The Examples demonstrate that causing a decrease in PDE10 activity is an effective method to reduce body weight and/or body fat, can ameliorate a symptom of metabolic syndrome, can be used, e.g., to treat patients (humans and companion animals) that are overweight, obese, and/or suffer one or more symptoms of metabolic syndrome, and to treat animal food stock species to produce leaner meat.

### Exemplary PDE10 Inhibitors

PDE10 inhibitors are known to those skilled in the art and may also be identified by standard assays known to those in the art, such as disclosed in Fawcett et al., Proc. Natl. Acad. Sci, 97: 3702-3707, 2000 (referred to as PDE11A), U.S. Patent Publication No. 2003/0032579, U.S. Patent Publication No. 2003/0096323, and as further described below.

The PDE10 inhibitors used in the methods of the invention include papaverine (U.S. Patent Publication No. 2003/0032579) and those disclosed in U.S. Non-Provisional patent application Ser. No. 11/062,133 filed on Feb. 18, 2005 and entitled “Tetrahydroisoquinolinyl Derivatives of Quinasoline and Isoquinoline.” Compounds disclosed as PDE10 inhibitors in the above-discussed U.S. Non-Provisional patent application are of the following Formula (I):

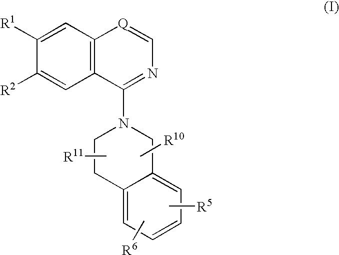

and to pharmaceutically acceptable salts, solvates and prodrugs thereof,

wherein Q is N or CH;

wherein R¹ and R² are each independently hydrogen, halogen, (C₁-C₉)alkyl, (C₂-C₉) alkenyl, (C₂-C₉) alkynyl, (C₃-C₈)cycloalkyl, —O—(C₁-C₉) alkyl, —O—(C₂-C₉) alkenyl, (C₁-C₆)alkyl O—(C₁-C₆)alkyl, —C≡N, —NO₂, —COOR³, —CONR³R⁴, —NR³R⁴, —COR⁴, or —OOH wherein said alkyl, alkenyl and alkynyl are optionally substituted with 1 to 3 halogens; wherein R³ and R⁴ are independently H, C₁-C₆ alkyl, or (C₂-C₆)alkenyl) wherein said alkyl and alkenyl are optionally substituted with from 1 to 3 halogen atoms; and, when R¹ and R² are independently —O— alkyl, —O-alkenyl, or alkyl, alkenyl or alkynyl, R¹ and R² may optionally be connected to form a 5 to 6 membered ring;

R⁵ and R⁶ are independently hydrogen; —C(O)—X, —SO₂—X, —N(Y)—O₂—X, or —N(Y)—C(O)—X, wherein X is a C₁-C₆ alkyl group unsubstituted or substituted with one or more halogens, —O—C₁-C₆ alkyl unsubstituted or substituted with one or more halogens, a (C₆-C₁₄) aryl group unsubstituted or substituted with one or two substituents, a —NR⁷R⁸ group or

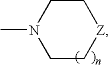

wherein said (C₆-C₁₄) aryl group substituents are independently selected from C₁-C₆ alkyl, —O—C₁-C₆ alkyl, halogen, —C≡N, —NO₂, —COOR³, —CONR³R⁴, —NR³R⁴, —COR³, and —COOH, and (C₁-C₆)alkyl substituted with 1 to 3 halogens;

Y is hydrogen or (C₁-C₆)alkyl;

n is 0 or 1;

R⁷ and R⁸ are each independently (C₁-C₆)alkyl or hydrogen;

Z is oxygen or NR⁹, wherein R⁹ is hydrogen or (C₁-C₆)alkyl;

wherein R¹⁰ and R¹¹ are independently H, halogen, C≡N, —COOH, —COOR³, —CONR³R⁴, COR³, —NR³R⁴, —OH, (C₆-C₁₄)aryl, 5 to 12 membered heteroaryl, (C₁-C₆)alkyl, (C₂-C₆) alkenyl, (C₂-C₆) alkynyl or (C₃-C₈) cycloalkyl wherein said alkyl, alkenyl, and alkynyl are optionally independently substituted with from 1 to 3 halogens;

A particular embodiment of the invention relates to compounds of the Formula (I) wherein Q is N and R¹ and R² are each —OCH₃.

Another embodiment of this invention relates to compounds of the Formula (I) wherein Q is N, R¹ and R² are each OCH₃ and one or both of R⁵ and R⁶ are —SO₂—X or —N(Y)—SO₂—X, wherein X and Y are as defined above.

A preferred embodiment of this invention relates to compounds of Formula (I) wherein Q is N, R¹ and R² are each —OCH₃ and one or both of R⁵ and R⁶ are —SO₂—X, where X is 4-methyl piperazine.

Another preferred embodiment of this invention relates to compounds of Formula (I) wherein Q is N, R¹ and R² are each —OCH₃ and one or both of R⁵ and R⁶ are —NH—SO₂—X, where X is mono- or disubstituted aryl. Preferably aryl is phenyl or naphthyl, optionally substituted with C₁-C₆ alkyl, C₁-C₆ alkoxy, —C≡N, —NO₂, —COOR³, —CONR³R⁴, —NR³R⁴, —COR³, and —COOH, wherein R³ and R⁴ are as defined above.

Another embodiment of this invention relates to compounds of Formula (I) wherein Q is N, R¹ and R² are each —OCH₃ and one or both of R⁵ and R⁶ is —N(Y)—C(O)—X, wherein X and Y are as defined above.

Another embodiment of the present invention relates to compounds of Formula (I) wherein Q is CH, R¹ and R² are each —OCH₃ and R⁵, R⁶, R¹⁰ and R¹¹ are hydrogen.

In another embodiment, R¹⁰ and R¹¹ are independently selected from hydrogen, (C₁-C₆)alkyl, (C₂-C₆)alkenyl, (C₂-C₆)alkynyl, and (C₃-C₈) cycloalkyl. In such embodiment, Q is preferably N.

Examples of specific compounds of the Formula (I) are the following:
N-[2-(6,7-dimethoxy-quinazolin-4-yl)-1,2,3,4-tetrahydro-isoquinolin-7-yl]-4-isopropyl-benzenesulfonamide;
N-[2-(6,7-dimethoxy-quinazolin-4-yl)-1,2,3,4-tetrahydro-isoquinolin-7-yl]-2,5-dimethyl-benzenesulfonamide; N-[2-(6,7-dimethoxy-quinazolin-4-yl)-1,2,3,4-tetrahydro-isoquinolin-7-yl]-2,2-dimethyl-propionamide; N-[2-(6,7-dimethoxy-quinazoline-4-yl)-1,2,3,4-tetrahydro-isoquinolin-7-yl]-acetamide; 4-chloro-N-[2-(6,7-dimethoxy-quinazolin-4-yl)-1,2,3,4-tetrahydro-isoquinolin-7-yl]-benzenesulfonamide; N-[2-(6,7-dimethoxy-quinazoline-4-yl)-1,2,3,4-tetrahydro-isoquinolin-7-yl]-acetamide; N-[2-(6,7-dimethoxy-quinazolin-4-yl)-1,2,3,4-tetrahydro-isoquinolin-7-yl]-4-ethyl-benzamide; 4-chloro-N-[2-(6,7-dimethoxy-quinazolin-4-yl)-1,2,3,4-tetrahydro-isoquinolin-7-yl]-benzamide; 3-chloro-N-[2-(6,7-dimethoxy-quinazolin-4-yl)-1,2,3,4-tetrahydro-isoquinolin-7-yl]-benzamide; 4-tert-butyl-N-[2-(6,7-dimethoxy-quinazolin-4-yl)-1,2,3,4-tetrahydro-isoquinolin-7-yl]-benzenesulfonamide; N-[2-(6,7-dimethoxyquinazolin-4-yl)-1,2,3,4-tetrahydro-isoquinolin-7-yl]4-ethoxy benzamide; N-[2-(6,7-dimethoxy-quinazolin-4-yl)-1,2,3,4-tetrahydro-isoquinolin-7-yl]-4-trifluoromethyl-benzenesulfonamide; N-[2-(6,7-dimethoxy-quinazolin-4-yl)-1,2,3,4-tetrahydro-isoquinolin-7-yl]-3,4-dimethoxy-benzenesulfonamide; 6,7-dimethoxy-4-[8-(morpholine-4-sulfonyl)-3,4-dihydro-1H-isoquinolin-2-yl]-quinazoline; 6,7-dimethoxy-4-[8-(4-methyl-piperazine-1-sulfonyl)-3,4-dihydro-1H-isoquinolin-2-yl]-quinazoline; 4-(7,8-dimethoxy-3,4-dihydro-1H-isoquinolin-2-yl)-6-ethoxy-7-methoxy-quinazoline; 4-(6,7-dimethoxy-3,4-dihydro-1H-isoquinolin-2-yl)-6-ethoxy-7-methoxy-quinazoline; 4-(6,7-dimethoxy-3-methyl-3,4-dihydro-1H-isoquinolin-2-yl)-6-ethoxy-7-methoxy-quinazoline; 4-(3,4-dihydro-1H-isoquinolin-2-yl)-6-ethoxy-7-methoxy-quinazoline; 4-(3,4-dihydro-1H-isoquinolin-2-yl)-7-methoxy-quinazoline; 2-(6,7-dimethoxy-quinazolin-4-yl)-1,2,3,4-tetrahydro-isoquinolin-7-ylamine; 6,7-dimethoxy-3′,4′-dihydro-1′H-[1,2′]biisoquinolinyl; and 6,7-dimethoxy-4-(3-propyl-3,4-dihydro-1H-isoquinolin-2-yl)-quinazoline.

A preferred compound from the above-listed compounds is 6,7-dimethoxy-4-[8-(morpholine-4-sulfonyl)-3,4-dihydro-1H-isoquinolin-2-yl]-quinazoline having the following structure (!A):

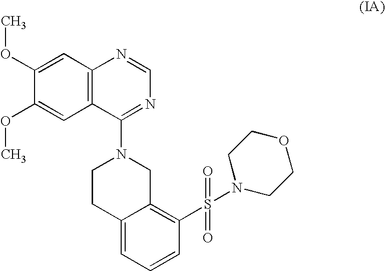

Other suitable PDE10 inhibitors include compounds disclosed in U.S. Provisional Application Ser. No. 60/640,405 filed on Dec. 31, 2004 and entitled “NOVEL PYRROLIDYL DERIVATIVES OF HETEROAROMATIC COMPOUNDS.” Compounds disclosed in this US Provisional include compounds having Formula (II)

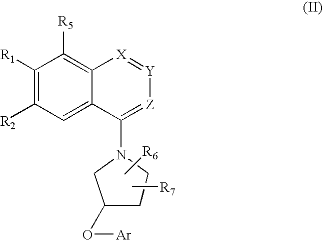

wherein X, Y and Z are each independently CH or N with the proviso that at least one or two of X, Y and Z are N, but not all three;

wherein R₁, R₂ and R₅ are independently H, halogen, C≡N, —COOH, —COOR₃, —CONR₃R₄, COR₃, —NR₃R₄, —NHCOR₃, —OH, (C₆-C₁₀)aryl, 5 to 7 membered heteroaryl, (C₁-C₆)alkyl, (C₂-C₆) alkenyl, (C₂-C₆) alkynyl, —O—(C₁-C₆) alkyl, —O—(C₂-C₆) alkenyl or (C₃-C₈) cycloalkyl; or, when R₁, R₂ and R₅ are independently —O—(C₁-C₆)alkyl, —O—(C₂-C₆) alkenyl, (C₁-C₆)alkyl, (C₂-C₆)alkenyl or (C₂-C₆) alkynyl, R₁ and R₂ or R₁ and R₅ may optionally be connected to form a 5 to 6 membered ring;

wherein R₃ and R₄ are independently H, (C₁-C₆)alkyl or (C₆-C₁₀)aryl said aryl optionally substituted with one or more (C₁-C₆)alkyl groups;

wherein R₆ and R₇ are each independently H, —COOR₃, —CONR₃R₄, —COR₄, NR₃R₄, —NHCOR₃, —OH, —(C₁-C₆)alkylene-OH, —HNCOOR₃, —CN, —HNCONHR₄, (C₁-C₆)alkyl, (C₂-C₆) alkoxy, C₆-C₁₀ aryl or

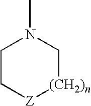

wherein n is 0 or 1;

Z is oxygen or NR₈, wherein R₈ is hydrogen or (C₁-C₆)alkyl;

wherein Ar is phenyl, naphthyl, or a 5 to 6-membered heteroaryl ring, which heteroaryl is optionally fused to a benzo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing phenyl, naphthyl, heteroaryl, or benzo-fused heteroaryl rings may optionally be substituted with from one to three substituents independently selected from (C₁-C₈) alkyl, chloro-, bromo-, iodo, fluoro-, (C₁-C₈)hydroxyalkyl-, (C₁-C₈)alkoxy-(C₁-C₈)alkyl-, (C₃-C₈)hydroxycycloalkyl-, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8 membered)heterocycloalkyl, wherein said alkyl, alkoxy and cycloalkyl may be optionally substituted with 1 to 3 halos and wherein each (C₃-C₈)cycloalkyl or heterocycloalkyl moiety may be independently substituted with from one to three (C₁-C₆)alkyl or benzyl groups; or

wherein Ar is a 5- to 6-membered heteroaryl ring, which heteroaryl is fused to an imidazo, pyrido, pyrimido, pyrazo, pyridazo, or pyrrolo group, and which heteroaryl contains from one to four heteroatoms selected from oxygen, nitrogen and sulfur, with the proviso that said heteroaryl ring cannot contain two adjacent oxygen atoms or two adjacent sulfur atoms, and wherein each of the foregoing fused heteroaryl rings may optionally be substituted with from one to three substituents independently selected from (C₁-C₈) alkyl, chloro-, bromo-, iodo, fluoro-, halo(C₁-C₈)alkyl, (C₁-C₈)hydroxyalkyl-, (C₁-C₈)alkoxy-(C₁-C₈)alkyl-, —O—(C₁-C₈)alkyl-halo, (C₃-C₈)hydroxycycloalkyl-, (C₃-C₈)cycloalkoxy-, (C₁-C₈)alkoxy-(C₃-C₈)cycloalkyl-, (3-8 membered)heterocycloalkyl, hydroxyl(3-8 membered)heterocycloalkyl, and (C₁-C₈)alkoxy-(3-8-membered)-heterocycloalkyl, wherein each (C₃-C₈)cycloalkyl or heterocycloalkyl moiety may be independently substituted with from one to three (C₁-C₆)alkyl or benzyl groups; or when Ar is phenyl, naphthyl, or heteroaryl ring, each ring may be optionally substituted with one to three substituents independently selected from (a) lactone formed from —(CH₂)ₜOH with an ortho —COOH, wherein t is one, two or three; (b) —CONR₁₄R₁₅, wherein R₁₄ and R₁₅ are independently selected from (C₁-C₈)alkyl and benzyl, or R₁₄ and R₁₅ together with the nitrogen to which they are attached form a 5- to 7-membered heteroalkyl ring that may contain from zero to three heteroatoms selected from nitrogen, sulfur and oxygen in addition to the nitrogen of the —CONR₁₄R₁₅ group, wherein when any of said heteroatoms is nitrogen it may be optionally substituted with (C₁-C₈)alkyl or benzyl, with the proviso that said ring cannot contain two adjacent oxygen atoms or two adjacent sulfur atoms; or (c) —(CH₂)ᵥNCOR₁₄R₁₅ wherein v is zero, one, two or three and —COR₁₄R₁₅ taken together with the nitrogen to which they are attached form a 4- to 6-membered lactam ring; a pharmaceutically acceptable salt thereof or a hydrate or solvate of the compound or the salt.

Suitable compounds having Formula (II) that may be used in the practice of the present invention include: 4-[3-Allyl-4-(quinoxalin-2-yloxy)-pyrrolidin-1-yl]-6,7-dimethoxy-quinazoline; 6,7-Dimethoxy-4-[3-propyl-4-(quinoxalin-2-yloxy)-pyrrolidin-1-yl]-quinazoline; 1-(6,7-Dimethoxy-quinazolin-4-yl)-3-methyl-4-(quinoxalin-2-yloxy)-pyrrolidine-3-carboxylic acid ethyl ester; 6,7-Dimethoxy-4-[3-methyl-3-(quinoxalin-2-yloxy)-pyrrolidin-1-yl]-quinazoline; [1-(6,7-Dimethoxy-quinazolin-4-yl)-4-quinoxalin-2-yloxy)-pyrrolidin-3-yl]-isopropyl-methyl-amine; [1-(6,7-Dimethoxy-quinazolinyl)-4-(quinoxalin-2-yloxy)-pyrrolidin-3-yl]-diethyl-amine; [1-(6,7-Dimethoxy-quinazolin-4-yl)-4-(quinoxalin-2-yloxy)-pyrrolidin-3-yl]-ethyl-methyl-amine; [1-(6,7-Dimethoxy-quinazolin-4-yl)-4-(quinoxalin-2-yloxy)-pyrrolidin-3-yl]-dimethyl-amine; [1-(6,7-Dimethoxy-quinazolin-4-yl)-4-quinolin-2-yloxy)-pyrrolidin-3-yl]-dimethyl-amine; [1-(6,7-Dimethoxy-quinazolin-4-yl)-4-(quinolin-3-yloxy)-pyrrolidin-3-yl]-dimethyl-amine; 6,7-Dimethoxy-4-[4′-(quinoxalin-2-yloxy)-[1,3′]bipyrrolidinyl-1′-yl]quinazoline; 6,7-Dimethoxy-4-[3-morpholin-4-yl-4-(quinoxalin-2-yloxy)-pyrrolidin-1-yl]-quinazoline; 6,7-Dimethoxy-4-[3-(4-methyl-piperazin-1-yl)-4-(quinoxalin-2-yloxy)-pyrrolidin-1-yl]-quinazoline; [1-(6,7-Dimethoxy-quinazolin-4-yl)-4-(quinoxalin-2-yloxy)-pyrrolidin-3-yl]-methyl-amine; N-[1-(6,7-Dimethoxy-quinazolin-4-yl)-4-(quinoxalin-2-yloxy)-pyrrolidin-3-yl]-N-methyl-acetamide; 6,7-Dimethoxy-4-[3-quinoxalin-2-yloxy)-pyrrolidin-1-yl]-quinazoline; 4-[3-(4-Ethoxy-phenoxy)-pyrrolidin-1-yl]-6,7-dimethoxy-quinazoline; 6,7-Dimethoxy-4-[3-(naphthalen-2-yloxy)-pyrrolidin-1-yl]-quinazoline; 4-[3-(4-tert-Butyl-phenoxy)-pyrrolidin-1-yl]-6,7-dimethoxy-quinazoline; 4-[1-(6,7-Dimethoxy-quinazolin-4-yl)-pyrrolidin-3-yloxy]-benzonitrile; 6,7-Dimethoxy-4-[3-(4-trifluoromethoxy-phenoxy)-pyrrolidin-1-yl]-quinazoline; 4-[3-(3-Ethoxy-phenoxy)-pyrrolidin-1-yl]-6,7-dimethoxy-quinazoline; 4-[3-(3,4-Dimethoxy-phenoxy)-pyrrolidin-1-yl]-6,7-dimethoxy-quinazoline;

4-[3-(3-Isopropoxy-phenoxy)-pyrrolidin-1-yl]-6,7-dimethoxy-quinazoline; 4-[3-(Indan-5-yloxy)-pyrrolidin-1-yl]-6,7-dimethoxy-quinazoline; 6,7-Dimethoxy-4-[3-(quinolin-6-yloxy)-pyrrolidin-1-yl]-quinazoline; N4-[3-(Biphenyl-3-yloxy)-pyrrolidin-1-yl]6,7-dimethoxy-quinazoline; 6,7-Dimethoxy-4-[3-(2-methyl-quinolin-6-yloxy)-pyrrolidin-1-yl]-quinazoline; 6,7-Dimethoxy-4-[3-(7-methoxy-naphthalen-2-yloxy)-pyrrolidin-1-yl]-quinazoline; 6,7-Dimethoxy-4-[3-(6-methoxy-naphthalen-2-yloxy)-pyrrolidin-1-yl]-quinazoline; 6,7-Dimethoxy-4-[3-(quinolin-7-yloxy)-pyrrolidin-1-yl]-quinazoline; 6,7-Dimethoxy-4-[3-(naphthalen-1-yloxy)-pyrrolidin-1-yl]-quinazoline; 4-[3-(Isoquinolin-3-yloxy)-pyrrolidin-1-yl]-6,7-dimethoxy-quinazoline; 4-[3-(Isoquinolin-7-yloxy)-pyrrolidin-1-yl]-6,7-dimethoxy-quinazoline; 6,7-Dimethoxy-4-[3-(pyridin-2-yloxy)-pyrrolidin-1-yl]-quinazoline; 6,7-Dimethoxy-4-[3-(pyridin-3-yloxy)-pyrrolidin-1-yl]-quinazoline; 6,7-Dimethoxy-4-[3-(pyridin-4-yloxy)-pyrrolidin-1-yl]-quinazoline; 4-[3-(5-Chloro-pyrimidin-2-yloxy)-pyrrolidin-1-yl]-6,7-dimethoxy-quinazoline; 3-[1-(6,7-Dimethoxy-quinazolin yl)-pyrrolidin-3-yloxy]-quinoxaline-6-carbonitrile acid tert-butyl ester; 6,7-Dimethoxy-4-[3-methoxy-4-(quinoxalin-2-yloxy)-pyrrolidin-1-yl]-quinazoline.tetrahydrofuran (pyrrolidin-3-yloxy]-quinoxaline; 1-(6,7-Dimethoxy-quinazolin-4-yl)-4-(quinoxalin-2-yloxy)-pyrrolidin-3-ol; [4-Benzyl-1-(6,7-dimethoxy-quinazolinyl)-pyrrolidin-3-yl]-dimethyl-amine; 6,7-Dimethoxy-4-[3-(quinoxalin-2-yloxy)-pyrrolidin-1-yl]-cinnoline; 6,7-Dimethoxy-4-[4′-(quinoxalin-2-yloxy)-[1,3′]bipyrrolidinyl-1′-yl]-cinnoline; [1-(6,7-Dimethoxy-cinnolin-4-yl)-4-(quinolin-2-yloxy)-pyrrolidin-3-yl]-ethyl-methyl-amine; [1-(6,7-Dimethoxy-cinnolin-4-yl)-4-(quinolin-2-yloxy)-pyrrolidin-3-yl]-diethyl-amine; 6,7-Dimethoxy-4-[3-morpholin-4-yl-4-(quinoxalin-2-yloxy)-pyrrolidin-1-yl]-cinnoline; [1-(6,7-Dimethoxy-cinnolin-4-yl)-4-(quinoxalin-2-yloxy)-pyrrolidin-3-yl]-diethyl-amine; [1-(6,7-Dimethoxy-cinnolin-4-yl)-4-(quinoxalin-2-yloxy)-pyrrolidin-3-yl]-ethyl-methyl-amine; [1-(6,7-Dimethoxy-cinnolin-4-yl)-4-(quinolin-2-yloxy)-pyrrolidin-3-yl]-dimethyl-amine; 6,7-Dimethoxy-4-[3-morpholin-4-yl-4-(quinolin-2-yloxy)-pyrrolidin-1-yl]-cinnoline; 6,7-Dimethoxy-4-[4′-(quinolin-2-yloxy)-[1,3′]bipyrrolidinyl-1′-yl]-cinnoline; 4-[3-(4a,5,6,7,8,8a-Hexahydro-quinoxalin-2-yloxy)-pyrrolidin-1-yl]6,7-dimethoxy-quinazoline; 1-(6,7-Dimethoxy-quinazolin-4-yl)-4-(quinoxalin-2-yloxy)-pyrrolidine-2-carboxylic acid dimethylamide; [1-(6,7-Dimethoxy-quinazolin-4-yl)-4-(quinoxalin-2-yloxy)-pyrrolidin-2-yl]-methanol hydrochloride; and 2-[1-(6,7-Dimethoxy-quinazolin-4-yl)-4-(quinoxalin-2-yloxy)-pyrrolidin-2-yl]-propan-2-ol hydrochloride. A preferred compound is 6,7-Dimethoxy-4-[3-(quinoxalin-2-yloxy)-pyrrolidin-1-yl]-quinazoline which may be prepared as described in the Examples (IIA) below. Other derivatives may be prepared using procedures analogous to this procedure.

Another set of PDE10 inhibitors that are useful in the practice of the present invention include quinazoline compounds of Formula (III) below.

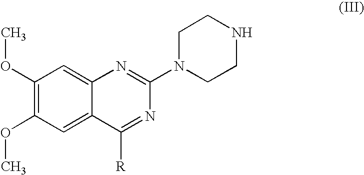

where R is a substituted phenyl; a pharmaceutically acceptable salt thereof, or a hydrate or solvate of the compound or the salt. A preferred compound of Formula (III) is 4-(2-Fluorophenyl)-6,7-dimethoxy-2-piperazin-1-yl-quinazoline which may be prepared as described in the Examples below (Compound IIIA). Other derivatives can be prepared using procedures analogous to those used to prepare Compound IIA in the Examples.

Another class of PDE10 inhibitors that are useful in the practice of the present invention include compounds disclosed in U.S. Provisional Ser. No. 60/642,058 filed on Jan. 7, 2005 and entitled “HETEROAROMATIC QUINOLINE COMPOUNDS.” Suitable compounds from this US Provisional include compounds of Formula (IV) below.

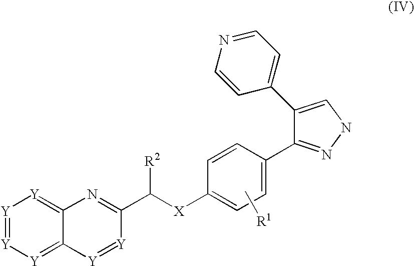

R¹ is hydrogen, halogen, hydroxyl, cyano, (C₁-C₈)alkyl, (C₂-C₈)alkenyl, (C₂-C₈)alkynyl, (C₁-C₈)alkoxy, halo-substituted (C₁-C₈)alkyl, (C₃-C₈)cycloalkyl, (C₂-C₇)heterocycloalkyl, (C₁-C₈)alkylthio, NR³R³, —O—CF₃, —S(O)ₙ—R³, C(O)—NR³R³, or (C₁-C₈)alkyl substituted with a nitrogen, oxygen or sulfur, where each R³ is each independently hydrogen, (C₁-C₈)alkyl, (C₂-C₈)alkenyl, (C₂-C₈)alkynyl, halo-substituted (C₁-C₈)alkyl, or (C₃-C₈)cycloalkyl, and n is 0, 1 or 2;

R² is hydrogen (C₁-C₈)alkyl, (C₂-C₈)alkenyl, or (C₂-C₈)alkynyl;

X is oxygen, sulfur, carbon or nitrogen wherein the carbon and nitrogen may be substituted with a substituent selected from (C₁-C₈)alkyl, (C₂-C₈)alkenyl, (C₂-C₈)alkynyl, or halo-substituted (C₁-C₈)alkyl;

Y is carbon or nitrogen, provided that not more than one Y is nitrogen, and when Y is carbon it is substituted with R⁷, where R⁷ is each independently selected from hydrogen, halogen, hydroxyl, cyano, (C₁-C₈)alkyl, (C₂-C₈)alkenyl, (C₂-C₈)alkynyl, (C₁-C₈)alkoxy, (C₁-C₈)cycloalkyl, (C₁-C₈)alkylthio, halo-substituted (C₃-C₈)alkyl, NR⁸R⁸, —O—CF₃, —S(O)ₘ—R⁸, and —C(O)—NR⁸R⁸, where each R⁸ is independently hydrogen or (C₁-C₈)alkyl and m is 0, 1 or 2; a pharmaceutically acceptable salt thereof or a hydrate or solvate of the compound or the salt.

Preferred compounds of Formula (IV) are 2-[4-(4-pyridin-4-yl-2H-pyrazol-3-yl)-phenoxymethyl]-quinoline which may be prepared as described in the Examples below (Compound IVA) and 2-[4-(1-Methyl-4-pyridinyl-1H-pyrazol-3-yl)-phenoxymethyl]-quinoline. Other derivatives may be prepared using procedures analogous to the procedures used to prepare Compound IVA in the Examples.

It will be understood by those skilled in the art that all stereoisomers, tautomers, solvates, hydrates, prodrugs, and pharmaceutically acceptable salts of the compounds listed above are also included as PDE10 inhibitors that can be used in the present invention.

As previously noted, other PDE10 inhibitors, including PDE10 selective inhibitors, can be identified using standard assays known to those skilled in the art. Briefly, one type of screen to identify PDE10 selective modulators uses native enzymes isolated from tissue or recombinant enzymes isolated from transfected host cells, for example, Sf9 insect cells (Fawcett, Proc. Natl. Acad. Sci. USA. 97: 3702-07, 2000), yeast cells (Loughney et al., U.S. Pat. No. 5,932,465), or COS-7 cells (Yuasa, J. Biol. Chem. 275: 31469-79, 2000). Preferably, the PDE10 enzyme is human (e.g., Loughney et al., U.S. Pat. No. 5,932,465, Lanfear et al., EP 967284), mouse (e.g., Lanfear et al., EP 967294), or rat (e.g., SEQ ID NO: 2).

PDE10 activity is measured, for example, as the rate of hydrolysis of an appropriate substrate, [³H]cAMP or [³H]cGMP. This activity is measured, for example, by SPA-based methods (Fawcett, Proc. Natl. Acad. Sci. USA 97: 3702-07, 2000; Phillips et al., WO 00/40733, and Thompson et al., Biochem. 18: 5228, 1979 (as modified using product code TRKQ7090/7100, Amersham Int'l Ltd., Buckhamshire, England)). Samples containing the PDE10 enzyme are contacted with a cAMP or cGMP substrate (Sigma Chemical), a portion (e.g., ¼ to ½) of which is ³H labeled (Amersham). Reactions are conducted in, for example, microtiter plates (e.g., Microfluor® plates, Dynex Technologies, Chantilly, Va.), and are terminated by the addition of yttrium silicate SPA beads (Amersham) containing excess unlabelled cyclic nucleotide. After the beads are allowed to settle in the dark, plates are read by a microtiter plate reader (e.g., TopCount®, Packard, Meriden, Conn.).

PDE10 activity may also be assayed by detection of ³²P-phosphate released from ³²P-cAMP or ³²P-cGMP (as described, for example, in Loughney et al., J. Biol. Chem. 271: 796-806, 1996, and Loughney, U.S. Pat. No. 5,932,465), or using antibodies to distinguish between the PDE substrates, cGMP or cAMP, and their hydrolyzed products (using, for example FlashPlate™ technology, NEN® Life Sciences Products, Boston, Mass.).

As an alternative to assaying PDE10 catalytic activity, agents can be identified as PDE10 positive modulators or negative modulators (antagonists) if they indirectly modulate PDE10 catalytic activity, for example, via post-translational modification (e.g., phosphorylation), modulation of allosteric ligand binding (e.g., via the GAF domain (Fawcett, Proc. Natl. Acad. Sci. USA 97: 3702-07, 2000)), or by binding to PDE10 themselves at either a catalytic or allosteric regulatory site. Methods for determining PDE10 phosphorylation and allosteric ligand binding are described in the literature (see, e.g., McAllister-Lucas et al., J. Biol. Chem. 270: 30671-79, 1995, and Corbin et al., Eur. J. Biochem. 267: 2760-67, 2000).

The test agents used for screening for PDE10 inhibitors may be selected individually or obtained from a compound library. Such agents include peptides, combinatorial chemistry-derived molecular libraries made of D- and/or L-configuration amino acids, phosphopeptides, antibodies, and small organic and inorganic compounds. Libraries include biological libraries, libraries of natural compounds, peptoid libraries (libraries of molecules having the functions of peptides, but with novel, non-peptide backbones which are resistant to enzymatic degradation yet remain bioactive) (see, e.g., Zuckermann, J. Med. Chem. 37: 2678-85, 1994), spatially addressable parallel solid phase or solution phase libraries, synthetic library methods requiring deconvolution, the “one-bead one-compound” library method, and synthetic library methods using affinity chromatography selection.

Examples of methods for the synthesis of molecular libraries can be found in the art, for example, in DeWitt et al., Proc. Natl. Acad. Sci. 90: 6909, 1993; Erd et al., Proc. Natl. Acad. Sci. 91: 11422, 1994; Zuckermann et al., J. Med. Chem. 37: 2678, 1994; Cho et al., Science, 261: 1303, 1995; Carrell et al., Angew. Chem. Int. Ed. Engl. 33: 2061, 1994; and Gallop et al., J. Med. Chem. 37: 1233, 1994.

Libraries of compounds may be presented in solution (e.g., Houghten, Biotechniques, 13: 412-421, 1992), or on beads (Lam, Nature 354: 82-841, 1991), on chips (Fodor, Nature 364: 555-556, 1993), bacteria or spores (Ladner, U.S. Pat. No. 5,223,409), plasmids (Cull et al., Proc. Natl. Acad. Sci. USA. 89: 1865-1869, 1992) or on phage (Scott et al., Science 249: 386-390, 1990; Devlin, Science 249: 404-406, 1990; Cwirla et al., Proc. Natl. Acad. Sci. (USA) 87: 6378-6382, 1990; Felici, J. Mol. Biol. 222: 301-310, 1991; Ladner, supra).

### Screening Methods

As is noted above, the invention also includes screening methods for identifying agents that can be used in the therapeutic methods described herein. These methods can include determination of whether an agent inhibits PDE10, followed by confirmation of it as being effective in reducing body weight, reducing body fat, or in treating a symptom of metabolic syndrome, as is noted above. Alternatively, the screening methods can simply involve testing agents that are known to be PDE10 antagonists for their efficacy in such therapeutic methods.

Testing an agent for its efficacy in inhibiting PDE10 activity can be carried out using methods that are well known in the art (see, e.g., Fawcett et al., Proc. Natl. Acad. Sci. 97: 3702-3707, 2000 and in U.S. Patent Publication No. 200310032579).

Therapeutic efficacy of such active compounds can be determined by standard therapeutic procedures in cell cultures or in animal models, e.g., for determining the ED50 (the concentration of compound that produces 50% of the maximal effect). Once an agent has been determined to be a PDE10 antagonist, or if a known PDE10 antagonist is being tested, the agent can then be tested to confirm that it is effective in the therapeutic methods described herein. Such testing can be carried out in appropriate animal model systems for the conditions described herein. For example, genetically obese mice (e.g., C57BL (ob/ob)), diet-induced obesity mice (i.e., DIO mice), or rats can be treated with a candidate agent and the effects of the agent on various parameters associated with the conditions described herein can be compared with those in animals that have been kept under similar conditions, with the exception of not being treated with the candidate agent. Parameters that can be tested for this purpose include, for example, body weight, body fat, insulin, glucose, triglycerides, free fatty acids, adiponectin, hemoglobin A1c, cholesterol, leptin and/or fructosamine. Examples of studies such as these are provided below in the Examples. Agents that are found to have a positive impact on these parameters can then be selected for testing in other pre-clinical or clinical studies, as can be determined by those of skill in this art.

### Characterizing PDE10 Antagonists

PDE10 antagonist agents that are found to have a positive impact on parameters relevant to the therapeutic methods discussed herein can be tested in pre-clinical or clinical studies, as can be determined by those of skill in this art.

The data obtained from cell culture assays and animal models can be used in formulating a range of dosage for use in humans. The dosage may vary depending upon the dosage form employed and the route of administration. For any compound used in the method of the invention, the therapeutically effective dose can be estimated initially from cell culture assays. A dose may be formulated in animal models to achieve a circulating plasma concentration range that includes the IC50. Such information can be used to more accurately determine useful doses in humans. Levels in plasma may be measured, for example, by high performance liquid chromatography.

### Therapeutic Methods

An agent identified as a PDE10 inhibitor is administered in a dose sufficient to reduce body weight or body fat, e.g., by reducing the mass of one or more adipose depots, or to ameliorate a symptom of metabolic syndrome. Such therapeutically effective amounts will be determined using routine optimization techniques that are dependent on, for example, the condition of the patient or animal, the route of administration, the formulation, the judgment of the practitioner, and other factors evident to those skilled in the art in light of this disclosure.

The PDE10 inhibitors suitable for use in accordance with the present invention can be administered alone but, in human therapy, will generally be administered in admixture with a suitable pharmaceutical excipient, diluent, or carrier selected with regard to the intended route of administration and standard pharmaceutical practice.

For example, the PDE10 inhibitors suitable for use in accordance with the present invention or salts or solvates thereof can be administered orally, buccally, or sublingually, in the form of tablets, capsules (including soft gel capsules), multi-particulate, gels, films, ovules, elixirs, solutions or suspensions, which may contain flavoring or coloring agents, for immediate-, delayed-, modified-, sustained-, dual-, controlled-release or pulsatile delivery applications. Such compounds may also be administered via fast dispersing or fast dissolving dosages forms or in the form of a high energy dispersion or as coated particles. Suitable pharmaceutical formulations may be in coated or un-coated form as desired.

Such solid pharmaceutical compositions, for example, tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), hydroxypropyl methylcellulose acetate succinate (HPMCAS), sucrose, gelatin and acacia. Additionally, lubricating agents such as magnesium stearate, stearic acid, glyceryl behenate and talc may be included.

Solid compositions of a similar type may also be employed as fillers in gelatin capsules or HPMC capsules. Preferred excipients in this regard include lactose, starch, cellulose, milk sugar, or high molecular weight polyethylene glycols. For aqueous suspensions and/or elixirs, the PDE10 inhibitor compounds may be combined with various sweetening or flavoring agents, coloring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, HPMC, HPMCAS, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form, i.e., within the matrix, and/or on the dosage form, i.e., upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used, i.e., in cases where the drug substance is insoluble, a fast dispersing dosage form can be prepared, and, in cases where the drug substance is soluble, a fast dissolving dosage form can be prepared.

The PDE10 inhibitors suitable for use in accordance with the present invention can also be administered parenterally, for example, intracavernosally, intravenously, intra-arterially, intraperitoneally, intrathecally, intraventricularly, intraurethrally, intrasternally, intracranially, intramuscularly or subcutaneously, or they may be administered by infusion or needle-free techniques. For such parenteral administration they are best used in the form of a sterile aqueous solution which may contain other substances, for example, enough salts or glucose to make the solution isotonic with blood. The aqueous solutions should be suitably buffered (preferably, to a pH of from about 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the PDE10 inhibitors for use in the present invention will usually be from 1 to 500 mg (in single or divided doses). A preferred dosage range is about 1 mg to about 100 mg. The dosage may by via single dose, divided daily dose, or multiple daily dose. Alternatively, continuous dosing, such as for example, via a controlled (e.g., slow) release dosage form can be administered on a daily basis or for more than one day at a time.

Thus, for example, tablets or capsules of the PDE10 inhibitors suitable for use in accordance with the present invention may contain from 1 mg to 250 mg of active compound for administration singly or two or more at a time, as appropriate. Preferred tablets or capsules will contain about 1 mg to about 50 mg of active compound for administration singly or two or more at a time, as appropriate. The physician in any event will determine the actual dosage which will be most suitable for any individual patient and it will vary with the age, weight and response of the particular patient. There can, of course, be individual instances where higher or lower dosage ranges are merited and such are within the scope of this invention.

The PDE10 inhibitors suitable for use in accordance with the present invention can also be administered intranasally or by inhalation and are conveniently delivered in the form of a dry powder inhaler or an aerosol spray presentation from a pressurized container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™ or 1,1,1,2,3,3,3-heptafluoropropane (HFA 227EA™), carbon dioxide or other suitable gas. In the case of a pressurized aerosol, the dosage unit may be determined by providing a valve to deliver a metered amount. The pressurized container, pump, spray or nebuliser may contain a solution or suspension of the active compound, e.g. using a mixture of ethanol and the propellant as the solvent, which may additionally contain a lubricant, e.g., sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a PDE10 inhibitor for delivery to the animal to be treated. The overall daily dose with an aerosol will be in the range of from 1 to 50 mg which may be administered in a single dose or, more usually, in divided doses throughout the day.

The PDE10 inhibitors suitable for use in accordance with the present invention may also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the PDE10 inhibitors suitable for use in accordance with the present invention can be administered in the form of a suppository or pessary, or they may be applied topically in the form of a gel, hydrogel, lotion, solution, cream, ointment or dusting powder. The PDE10 inhibitors suitable for use in accordance with the present invention may also be dermally or transdermally administered, for example, by the use of a skin patch. They may also be administered by the pulmonary or rectal routes.

The PDE10 inhibitors may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the PDE10 inhibitors suitable for use in accordance with the present invention can be formulated as a suitable ointment containing the active ingredient or agent suspended or dissolved in, for example, a mixture with one or more of the following: mineral oil, liquid petrolatum, white petrolatum, propylene glycol, polyoxyethylene polyoxypropylene compound, emulsifying wax, and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, and water.

The PDE10 inhibitors suitable for use in accordance with the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are some of the most commonly used and suitable examples are described in PCT Publication Nos. WO 91/11172, WO 94/02518 and WO 98/55148.

Generally, in humans, oral administration is the preferred route, often being the most convenient. In circumstances where the recipient suffers from a swallowing disorder or from impairment of drug absorption after oral administration, the drug may be administered parenterally, sublingually, or buccally.

For veterinary use, a PDE10 inhibitor is administered as a suitably acceptable formulation in accordance with normal veterinary practice and the veterinary surgeon will determine the dosing regimen and route of administration which will be most appropriate for a particular animal. Such animals include companion animals who are overweight, obese, or at risk of being overweight or obese. Other animals that may be treated according to the present invention are foodstock animals in order to obtain leaner meat than would be obtained absent treatment according to the present invention.

The PDE10 inhibitors used in accordance with the present invention may also be used in conjunction with other pharmaceutical agents for the treatment of the diseases, conditions and/or disorders described herein. Therefore, methods of treatment that include administering PDE10 inhibitors in combination with other pharmaceutical agents are also provided. Suitable pharmaceutical agents that may be used in combination with the compounds of the present invention include anti-obesity agents such as apolipoprotein-B secretion/microsomal triglyceride transfer protein (apo-B/MTP) inhibitors, 11β-hydroxy steroid dehydrogenase-1 (11β-HSD type 1) inhibitors, peptide YY₃₋₃₆ or analogs thereof, MCR-4 agonists, cholecystokinin-A (CCK-A) agonists, monoamine reuptake inhibitors (such as sibutramine), cannabinoid receptor-1 antagonists (such as rimonabant), sympathomimetic agents, β₃ adrenergic receptor agonists, dopamine agonists (such as bromocriptine), melanocyte-stimulating hormone receptor analogs, 5HT2c agonists, melanin concentrating hormone antagonists, leptin (the OB protein), leptin analogs, leptin receptor agonists, galanin antagonists, lipase inhibitors (such as tetrahydrolipstatin, i.e. orlistat), anorectic agents (such as a bombesin agonist), neuropeptide-Y receptor antagonists (e.g., NPY Y5 receptor antagonists, such as the spiro compounds described in U.S. Pat. Nos. 6,566,367; 6,649,624; 6,638,942; 6,605,720; 6,495,559; 6,462,053; 6,388,077; 6,335,345; and 6,326,375; US Pat. Appl. Publ. Nos. 2002/0151456 and 2003/036652; and PCT Publication Nos. WO 03/010175, WO 03/082190 and WO 02/048152), thyromimetic agents, dehydroepiandrosterone or an analog thereof, glucocorticoid receptor agonists or antagonists, orexin receptor antagonists, glucagon-like peptide-1 receptor agonists, ciliary neurotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or inverse agonists, neuromedin U receptor agonists and the like. Other anti-obesity agents, including the preferred agents set forth hereinbelow, are well known, or will be readily apparent in light of the instant disclosure, to one of ordinary skill in the art.

Especially preferred are anti-obesity agents selected from the group consisting of orlistat, sibutramine, bromocriptine, ephedrine, leptin, rimonabant, pseudoephedrine; peptide YY₃₋₃₆ or an analog thereof; and 2-oxo-N-(5-phenylpyrazinyl)spiro-[isobenzofuran-1(3H), 4′-piperidine]-1′-carboxamide. Preferably, compounds of the present invention and combination therapies are administered in conjunction with exercise and a sensible diet.

Representative anti-obesity agents for use in the combinations, pharmaceutical compositions, and methods of the invention can be prepared using methods known to one of ordinary skill in the art, for example, sibutramine can be prepared as described in U.S. Pat. No. 4,929,629; bromocriptine can be prepared as described in U.S. Pat. Nos. 3,752,814 and 3,752,888; orlistat can be prepared as described in U.S. Pat. Nos. 5,274,143; 5,420,305; 5,540,917; and 5,643,874; rimonabant can be prepared as described in U.S. Pat. No. 5,624,941; PYY₃₋₃₆ (including analogs thereof) can be prepared as described in US Publication No. 2002/0141985 and PCT Publication No. WO 03/027637; and the NPY Y5 receptor antagonist 2-oxo-N-(5-phenylpyrazinyl)spiro[isobenzofuran-1(3H),4′-piperidine]-1′-carboxamide can be prepared as described in U.S. Patent Publication No. 2002/0151456.

### Kits

The invention also provides kits or pharmaceutical packages that include PDE10 antagonists for use in the prevention and treatment of the diseases and conditions described herein. In addition to one or more PDE10 antagonist in the form of, for example, tablets, capsules, or lyophilized powders, the kits or packages can include instructions for using the antagonists in the prevention or treatment of such diseases and conditions. The antagonists can be provided in the kits or packages in a bottle or another appropriate form (e.g., a blister pack). Optionally, the kits or pharmaceutical packages can also include other pharmaceutically active agents (see, e.g., the agents listed above, such as anti-obesity agents), and/or materials used in administration of the drug(s), such as diluents, needles, syringes, applicators, and the like.

The invention is based, in part, on the following experimental results. While the invention is described herein in connection with specific embodiments, it will be understood that other changes and modifications that can be practiced are also part of this invention and are also within the scope of the appended claims. This application is intended to cover any equivalents, variations, uses, or adaptations of the invention that follow, in general, the principles of the invention, including departures from the present disclosure that come within known or customary practice within the art, and that are able to be ascertained without undue experimentation.

### EXAMPLES

The compounds used in the following methods were prepared as described herein below.

Rimonabant (also known under the tradename ACOMPLIA) is available from Sanofi-Synthelabo Inc., New York, N.Y.; or prepared as described in U.S. Pat. No. 5,624,941

### Preparation of 6,7-Dimethoxy-4-[3-(quinoxalin-2-yloxy)-pyrrolidin-1-yl]-quinazoline

### Compound IIA

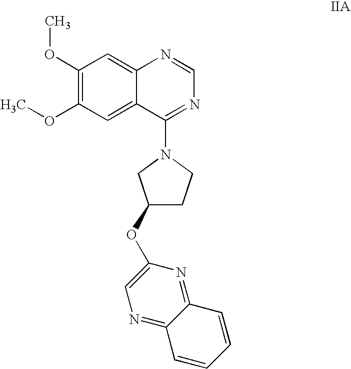

A mixture of 2-(pyrrolidin-3-yloxy)-quinoxaline (10.5 g, 48.9 mmol), 6,7-dimethoxy-4-chloroquinazoline (11.0 g, 48.9 mmol), and potassium carbonate (33.8 g, 244 mmol) in THF (500 mL) and water (100 mL) was heated to 75° C. for 16 hours. The mixture was concentrated under vacuum and the residue was dissolved in methylene chloride. The solution was washed with 1N NaOH and dried through a cotton plug. The solution was then concentrated and the resultant brown foam was chromatographed on silica gel eluting with 9:1 ethyl acetate/methanol to provide 17.3 g of an off-white powder. Trituration with ethyl acetate/methanol gave 15.0 g of an off-white powder. The solid was suspended in isopropanol and treated with 1.0 molar equivalents of sulfuric acid. After stirring overnight, the solids were collected via filtration to give 18.0 g of the title compound (IIA) as a white powder. Mass spectrum (M+H) m/z=404.2.

### Preparation of 4-(2-Fluorophenyl)-6,7-dimethoxy-2-piperazin-1-yl-quinazoline

### IIIA

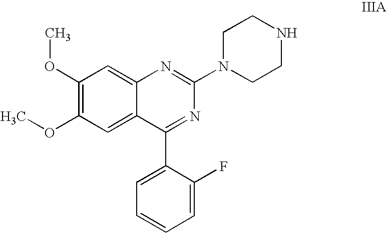

Step 1: To solution of 2,4-dichloro-6,7-dimethoxy-quinazoline (555 mg, 2.14 mmol) in dioxane (3.6 ml, 0.5M) was added 2-fluoro phenyl boronic acid (250 mg, 1.78 mmol), tricyclohexyl phosphine (30 mg, 0.06 eq.), Cesium carbonate (867 mg, 1.5 eq.) and tris(dibenzylideneacetone)dipalladium (32 mg, 0.02 eq.). The reaction mixture was heated at 100° C. under N₂ for 18 hours. The reaction mixture was filtered through celite and concentrated. MPLC purification using a biotage 40M column eluting with 5-40% ethyl acetate/hexans afforded the title compound as a white solid (333 mg, 59%).

Final Step: To a solution of 2-chloro-4-(2-fluorophenyl)-6,7-dimethoxyquinazoline (287 mg, 0.9 mmol) in ethanol (2 ml, 0.5 M) was added piperazine (386 mg, 5 eq.). The reaction mixture was heated at 100° C. for 18 hours. Reaction mixture was concentrated and purified via MPLC using a biotage 25M column eluting with 0-15% methanol/chloroform with 0.5% ammonium hydroxide to provide the title compound as a yellow solid (231 mg, 71%). Acc. Mass: (APCI m/z=369.1742 for C₂₀H₂₂N₄O₂F MW calculated 369.1727)

### Preparation of 2-[4-(4-Pyridin-4-yl-2H-pyrazol-3-yl)-phenoxymethyl]-quinoline

### Compound IVA

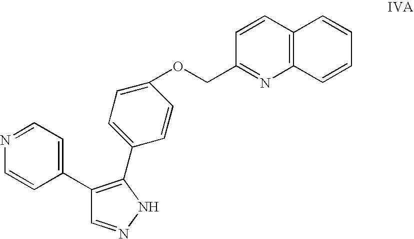

Step 1: To a solution of 2-Chloromethyl quinoline (2 g, 9.3 mmole) in acetone (47 ml, 0.2M) was added 4-hydroxy benzoic acid methyl ester (1.42 g, 1.0 eq.) and potassium carbonate (3.86 g, 3 eq.). The reaction mixture was heated at 60° C. for 16 hours under N₂ atmosphere, cooled to ambient temperature and poured into 1N sodium hydroxide (50 ml)/ethyl acetate (100 ml). The layers were separated and the organic layer dried magnesium sulfate, filtered and concentrated. Biotage MPLC was run using a 5-30% ethyl acetate/hexane gradient on a 40 M column to provide the title compound as a white solid (1.66 g, 61%). ¹H NMR (400 MHz, CDCl₃) δ 8.18 (d, J=8.7 Hz, 1H), 8.07 (d, J=8.3 Hz, 1H), 7.95 (M, 2H), 7.82 (d, J=7.9 Hz, 1H), 7.74 (dt, J=7.1, 1.7 Hz, 1H), 7.62 (d, J=8.3 Hz, 1H), 7.55 (dt, J=7.9, 1.2 Hz, 1H), 7.03 (d, J=9.1, 2H), 5.41 (s, 2H), 3.84 (s, 3H); MS: (M⁺H m/z=294.2)

Step 2: To a solution of 4-(Quinolin-2-ylmethoxy)-benzoic acid methyl ester (500 mg, 1.7 mmole) in tetrahydrofuran (8.5 ml) and methanol (3 ml) was added 1N NaOH (3.4 ml, 2 eq.). The reaction mixture was stirred at ambient temperature for 16 hours. To the reaction mixture was added 50 ml of brine and the pH was adjusted to 3 with 1N HCl to provide a white precipitate which was filtered and dried to provide the title compound as a white solid (463 mg, 98%). ¹H NMR (400 MHz, DMSO) δ 8.39 (d, J=8.3 Hz, 1H), 7.99 (m, 2H), 7.81 (M, 2H), 7.76 (dt, J=8.3, 1.7 Hz, 1H), 7.64 (d, J=8.3 Hz, 1H), 7.60 (dt, J=7.9, 1.3 Hz, 1H), 7.12 (M, 2H), 5.41 (s, 2H); MS: (M⁺H m/z=280.2)

Step 3: To a solution of 4-(Quinolin-2-ylmethoxy)-benzoic acid (25.98 g, 93 mmole) was added 250 ml of thionyl chloride under N₂. The reaction mixture stirred 3 hours and the excess thionyl chloride was removed under vacuum. The acid chloride was dissolved in tetrahydrofuran (450 ml) and triethylamine (50 ml, 4 eq.) was slowly added. O,N-dimethyl hydroxyl amine hydrochloride (27 g, 3 eq.) was added and the reaction stirred 18 hours. The reaction mixture was placed on a rotoevaporator to remove the solvent, partitioned between 1N NaOH and methylene chloride, separated, dried magnesium sulfate, filtered and concentrated. The crude product was filtered through silica gel eluting with 30-70% ethyl acetate/hexane to proved the title compound as a brown oil (26.26 g, 87%); ¹H NMR (400 MHz, CDCl₃) δ 8.17 (d, J=8.7 Hz, 1H), 8.06 (d, J=8.3 Hz, 1H), 7.81 (d, J=8.3 Hz, 1H), 7.67 (m, 3H), 7.63 (d, J=8.3 Hz, 1H), 7.52 (m, 1H), 7.01 (M, 2H), 5.39 (s, 2H), 3.52 (s, 3H) 3.31 (s, 2H); MS: (M⁺H m/z=323.2)

Step 4: To a solution of Lithium diisopropyl amide (1.0M) in tetrahydrofuran was added 4-picoline dropwise (7.55 ml, 5 eq.) at 0° C. under N₂. After 30 minutes the anion was cooled to −78° C. In a separate round bottom flask N-Methoxy-N-methyl-4-quinolin-2-ylmethoxy)-benzamide (5.0, 15.5 mmole) was dissolved in tetrahydrofuran (77 ml, 0.2M) and cooled to −78° C. under N₂. 1.2 eq. of the 4-picoline anion was added dropwise to the amide solution. After 45 min, 1 eq. more of the 4-picoline anion was added. After an addition 30 min, acetic acid (40 ml) was added dropwise and the reaction was slowly warmed to ambient temperature. The solid product (acetate salt) was filtered and partitioned between saturated sodium bicarbonate and dichloromethane. The layers were separated, dried magnesium sulfate filtered and concentrated to provide the title compound as a tan solid (4.41 g, 80%). ¹H NMR (400 MHz, CDCl₃) δ 8.52 (d, J=5.8 Hz, 2H), 8.19 (d, J=8.7 Hz, 1H), 8.07 (d, J=8.7 Hz, 1H), 7.93 (m, 2H), 7.82 (d, J=8.3 Hz, 1H), 7.75 (m, 1H), 7.61 (d, J=8.3 Hz, 1H), 7.54 (dt, J=7.9, 1.0 Hz, 1H), 7.23 (m, 2H) 7.07 (m, 2H), 5.42 (s, 2H), 4.19 (s, 2H); MS: (M⁺H m/z=355.2)

Step 5: To 2-pyridin-4-yl-1-[4-quinolin-2-ylmethoxy)-phenyl]-ethanone (4.0 g, 11.3 mmole) was added dimethoxymethyl-dimethyl amine (10 ml) and the reaction mixture was heated at reflux for 1 hour. Concentrated to give a quantitative yield of the title compound which was used as is in the next step. LC/MS: RT=1.4 min, MS: (M⁺H m/z=410.2)

Final step: To a solution of 3-Dimethylamino-2-pyridin-4-yl-1-[4-(quinolin-2-ylmethoxy)-phenyl}-propenone (9.57 g, 27 mmole) in methanol was added hydrazine hydrate (3.33 g, 40.5 mmole) and the reaction mixture was heated at reflux for 1 hour. The solvent was evaporated to yield a white solid. The solid was washed with water and ethyl ether. The solid was recrystallized from hot ethanol/ethylacetate (10 ml/g) to give 8.34 g of the title compound (IVA: 82%). ¹H NMR (400 MHz, DMSO) δ 8.41 (m, 3H), 8.16 (s, 1H), 7.97 (m, 2H), 7.86 (s, 1H), 7.75 (t, J=7.9 Hz, 1H), 7.68 (d, J=8.3 Hz, 1H), 7.60 (t, J=7.5 Hz, 1H), 7.33 (m, 2H), 7.18 (m, 2H) 7.15 (d, J=8.3 Hz, 1H), 7.06 (d, J=8.3 Hz, 1H), 5.38 (d, J=13.7 Hz, 2H); MS: (M⁺H m/z=379.2).

### Methods

Age matched male PDE10 KO (−/−) mice (U.S. Patent Publication No. 2003/0121069) (n=20) and wild type (+/+) littermate controls (n=20) were allowed to acclimate for two weeks prior to the start of the study and were given free access to water and Purina 5001 rodent chow (Purina, Brentwood, Mo.).

Mice were individually housed and divided into two groups: a group of control mice which remained on Purina 5001 chow; and another group which was switched to a diet composed of 45 kcal % fat (D12331 Rodent Diet, Research Diets, Inc., New Brunswick, N.J.) for the duration of the 6 week study. Food intake was measured on a 24-hour, 5 days/week cycle (Monday-Friday). Body weight was determined on Day 0 and thereafter at the same time that food intake was measured.

Body composition was analyzed on Day 0 and after the 6^{th} week of the study using PIXImus™ instrumentation (GE Lunar Corporation, Madison, Wis.). To assess the adipose depot mass, 360° radioscopic images of the mice were obtained using a commercially available micro computed tomography (CT) system (MicroCAT®, ImTek Inc., Oak Ridge, Tenn.) with a high-resolution CCD/phosphor screen detector. The scanner consisted of a cylindrical diameter/long field view of 36 mm/36 mm with a spatial resolution of less than 50 μM. The X-ray source was biased at 40 KeV with the anode current set to 0.4 mA. Anesthetized mice were placed on a radiotransparent mouse bed in an anatomically correct supine position, caudal end closest to the micro CT with the rostral end held in place against an anesthesia delivery tube. An initial radiographic image was acquired at 90° to the plane of the mouse bed to allow correct positioning of the mouse by centering the scan acquisition area at the level of the iliac crest of each mouse. Once correct alignment was assured, each animal was scanned. Each scan consisted of 196 individual projections with an exposure time of 250 μs/projection; total image acquisition time was approximately 12 minutes at 145 μM resolution.

Image reconstruction, whereby the 196 projections acquired in the micro CT scan of the mouse were manipulated to produce two-dimensional cross sectional images of the mouse, was performed using the MicroCAT® Reconstruction, Visualization, and Analysis Software (ImTek Inc., Oak Ridge, Tenn.) (Paulus et al., Neoplasia 2: 62-70, 2000). Two sets of reconstructed images per scan were generated for each mouse for the determination of individual fat depot mass. The first set of six reconstructed images provided a montage for the analysis of inguinal and epididymal adipose tissue depot mass. The second reconstruction set consisted of nine slices, determined by both intervertebral and midvertebral landmarks, and was used to determine retroperitoneal and mesenteric adipose tissue depot mass.

For image analysis, reconstructed bitmap images were converted to TIFF images. The TIFF images were subsequently analyzed and fat depot mass determined using Scion Image for Windows® (Scion Corporation, Frederick Md.). Demarcation lines separating individual fat depots were placed using the paintbrush tool (pixel size #3) and total pixel counts of each adipose region determined by the Scion Image software. An upper and lower pixel intensity threshold was chosen, in this study, a look-up-table (LUT) of between 115-187 was determined to be optimal for capturing the adipose depot.

Average pixel number between each slice was calculated (sliceₙ+sliceₙ₊₁)/2). Total pixel number, representing the individual fat depots, was calculated by multiplying the average pixel number between each slice by the average pixel number of each slice. Finally, the pixel count was converted into depot mass with the following equation: Depot mass (mg)=0.000915 g/ul×0.000757 μl/voxel×1000 mg/g×voxel count. The first factor corrects for specific gravity of glyceryl trioleate, representative of the density of the primary storage form of lipid in adipose tissue, i.e., triglyceride. The second factor is the volume per pixel and the third factor converts the resulting mass into mg units.

Following determination of body composition, energy expenditure and oxygen consumption were determined using an Oxymax system (Columbus Instruments, Columbus, Ohio). Mice were housed under standard laboratory conditions and maintained on the experimental diet. Mice were acclimated to sealed chambers (8″×4″×5.5″) of the calorimeter (one mouse per chamber). The chambers were placed in activity monitors. The calorimeter was calibrated before each use, airflow was adjusted to 1.6 liters/min, and the system setting and sampling times were set to 60 seconds and 15 seconds, respectively. Oxygen consumption, carbon dioxide production, and ambulatory activity were measured every 10 minutes for a period of 4 hours.

An oral glucose tolerance test was conducted after the end of the 8^{th} week of the study on mice around 8:30 am following an overnight fast. Retro-orbital blood samples were collected at time zero and then a 2 g/kg body weight oral glucose load was administered. Additional blood samples were collected at 30, 75, and 120 minutes post-glucose challenge. 25 μL of blood was added to 100 μL of 0.025 percent heparinized-saline in microtubes (Denville Scientific, Inc., Metuchen, N.J.). The tubes were spun at the highest setting in a Microfuge® 12 (Beckman Coulter, Fullerton, Calif.) for 2 minutes. Plasma was collected for plasma glucose determination, as further described below.

On the morning of the last day of the study, body weights and core body temperatures were determined and then blood samples were taken via retro-orbital sinus for plasma glucose, triglycerides, cholesterol, and free fatty acid determination. The mice were then sacrificed and about one milliliter of blood was collected in Microtainer® plasma separator tubes with lithium heparin (Becton-Dickinson, Inc., Franklin Lakes, N.J.). The tubes were spun in a Beckman Microfuge 12 at the maximum setting for five minutes. Plasma was collected in 1.5 ml Eppendorf tubes and frozen in liquid nitrogen. Epididymal fat pads were also removed, weighed, and snap frozen in liquid nitrogen. Biopsies of liver and muscle were also collected. All samples stored at −80° C.

Plasma glucose, triglycerides, cholesterol, hemoglobin A1c, and fructosamine were measured using the Roche/Hitachi 912 Clinical Chemistry Analyzer and manufacturer kit supplies (Roche Diagnostics Corp., Indianapolis, Ind.). Plasma free fatty acids (FFA) were measured using the above-described analyzer with a NEFA C kit (Wako Chemicals USA, Richmond, Va.). Plasma cGMP was measured using the BioTrak™ enzyme-immunoassay system (Amersham, Piscataway, N.J.). Plasma insulin and leptin were assessed via a similar technique using the Mercodia ELISA Insulin kit and mLeptin ELISA kit, respectively; both were supplied by ALPCO Diagnostics (Windham, N.H.). Plasma adiponectin was quantitated using a mouse adiponectin ELISA kit (Linco Research, St Charles, Mo.). All assays were conducted according to each manufacturer's instructions.

### Oxygen Consumption

For testing the effects of a PDE10 inhibitor on body weight and oxygen consumption, a mouse model of diet-induced obesity (DIO) was used. Obesity was induced in the mice by maintaining them on a 45% fat diet for more than 14 weeks. DIO mice were treated with Compound IVA at a dose of 15 mg/kg, administered twice daily (p.o.) for 14 days. Control mice were administered a vehicle of 0.5% methylcellulose. Measurements were made as previously described.

### Fasted-Induced Feeding Assay

PDE10 inhibitors were also tested for efficacy in reducing the acute re-feeding response following food withdrawal. Male CD rats were fasted overnight. Rats were administered a PDE10 inhibitor (Compound IIA and IIIA in separate tests) 30 minutes prior to the return of food. Food consumption was monitored and weighed at predetermined time points and the effect of PDE10 inhibitors were compared to vehicle-treated control rats.

### Example 1

### Effect of PDE10 Inhibition on Body Weight, Body Fat, and Metabolic Rate in Mice Fed a High Fat Diet

### KO Mice

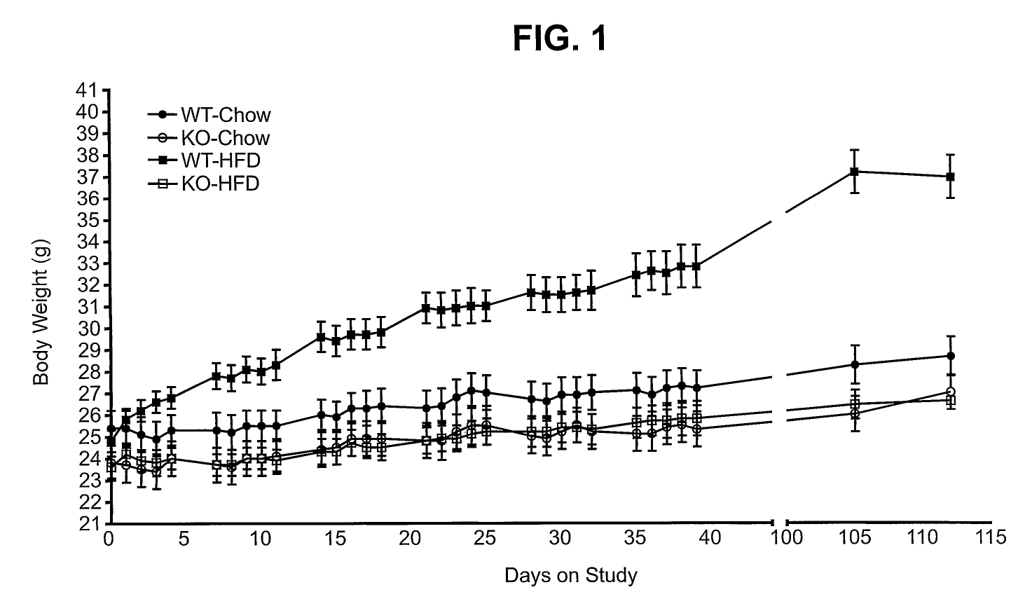

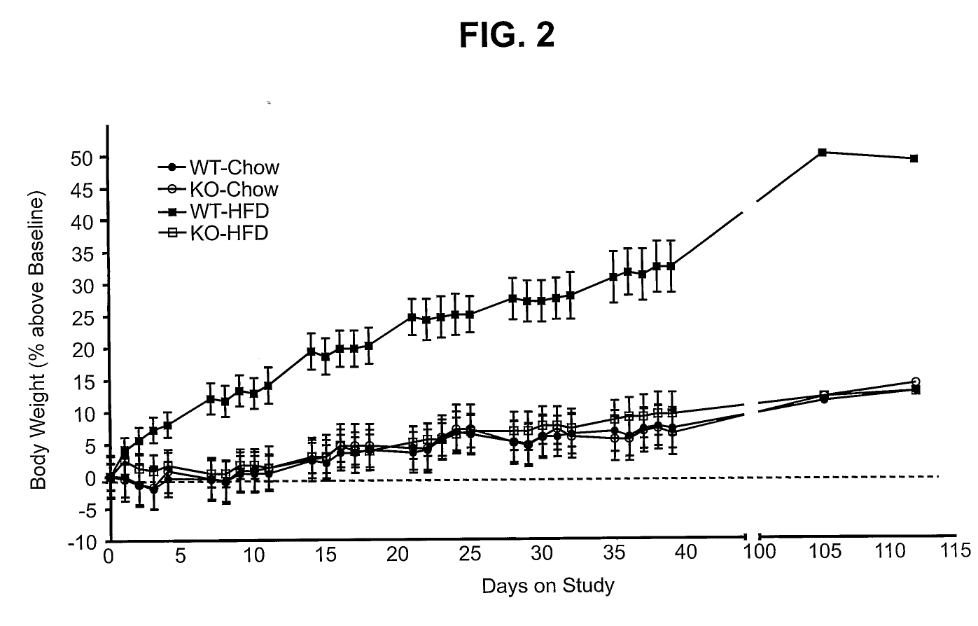

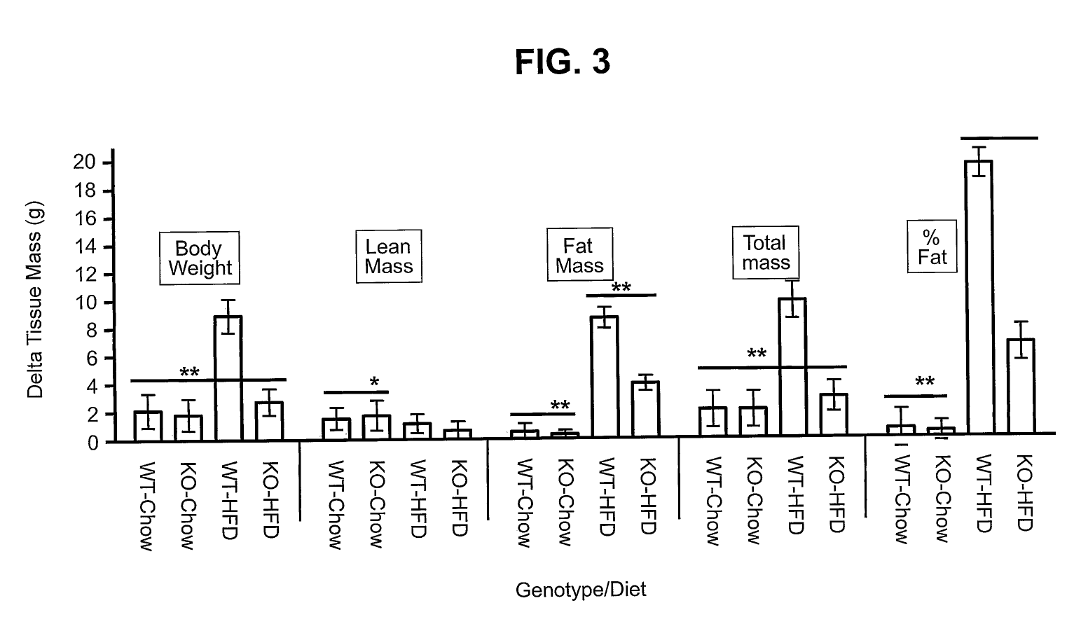

PDE10 inhibition, as a result of PDE10 gene disruption in the PDE10 KO mice, resulted in a robust phenotype of resistance to developing obesity while consuming a HFD. As shown in FIG. 1, wild type mice on the HFD demonstrated an increase in body weight as compared to the chow-fed wild type mice whereas HFD-fed PDE10 KO maintained a body weight comparable to the chow-fed PDE10 KO mice. When body weight was calculated as the percent of baseline weight, the obesity-resistant phenotype of the HFD-fed PDE10 KO mice remains clearly evident, as shown in FIG. 2. In contrast to the HFD-fed wild type mice, exhibiting an increase in body weight of approximately 30-35% above baseline weight in just 7 weeks, the HFD-fed PDE10 KO mice increased only 5-10% above their baseline weight, an increase comparable to that observed in both the chow-fed PDE10 KO mice and the chow-fed wild type mice. The body composition results shown in FIG. 3 demonstrate that the weight gain experienced by the HFD-fed wild type mice was due to increased adiposity. While there was some gain in body fat in the HFD-fed PDE10 KO mice, it was dramatically less compared to the HFD-fed wild type group (7% vs. 19% of total body weight, respectively).

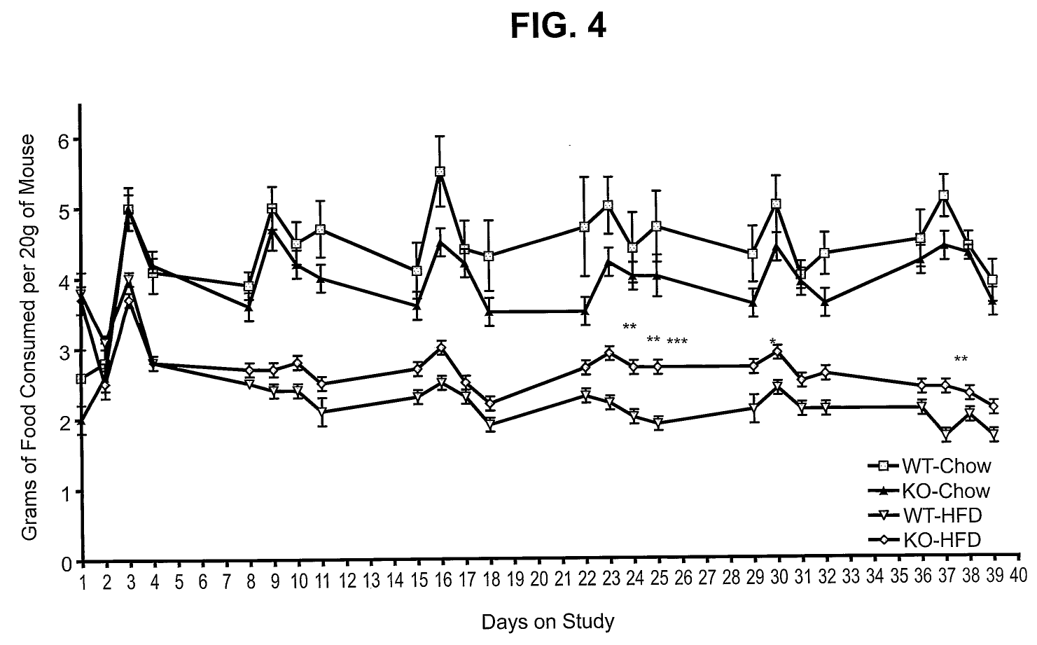

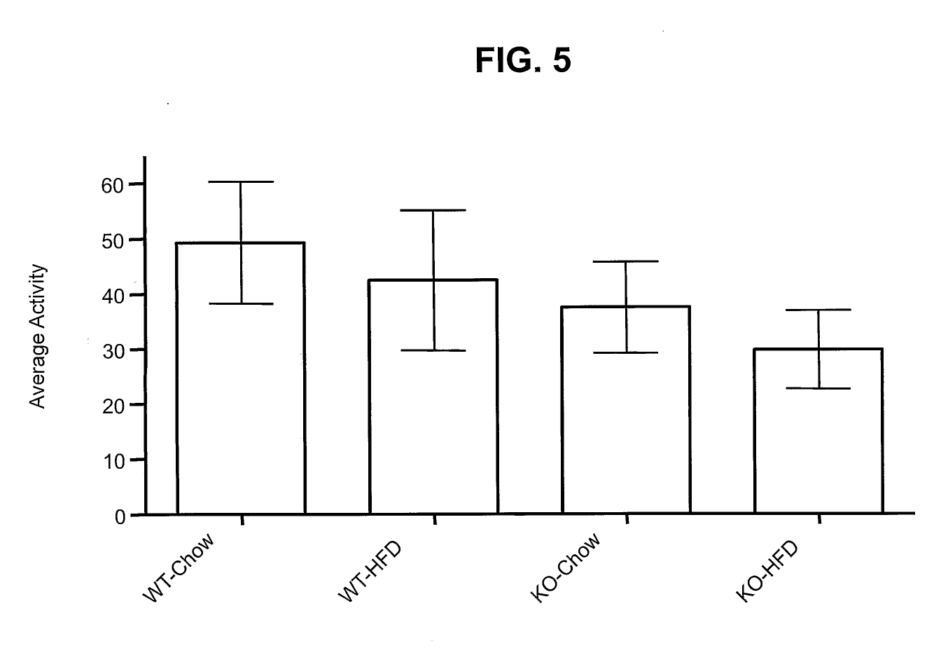

The differences in body weight between the HFD-fed wild type and HFD-fed PDE10 KO mice could not be explained by either reduced food consumption or increased activity level in the PDE10 KO mice. PDE10 KO mice consumed the HFD to an equal degree, or possibly even a greater degree, as compared to the wild type counterparts (FIG. 4). In addition, the PDE10 KO mice did not display any increase in activity level. To the contrary, the PDE10 KO mice actually displayed a trend towards reduced activity as compared to the wild type mice (FIG. 5).

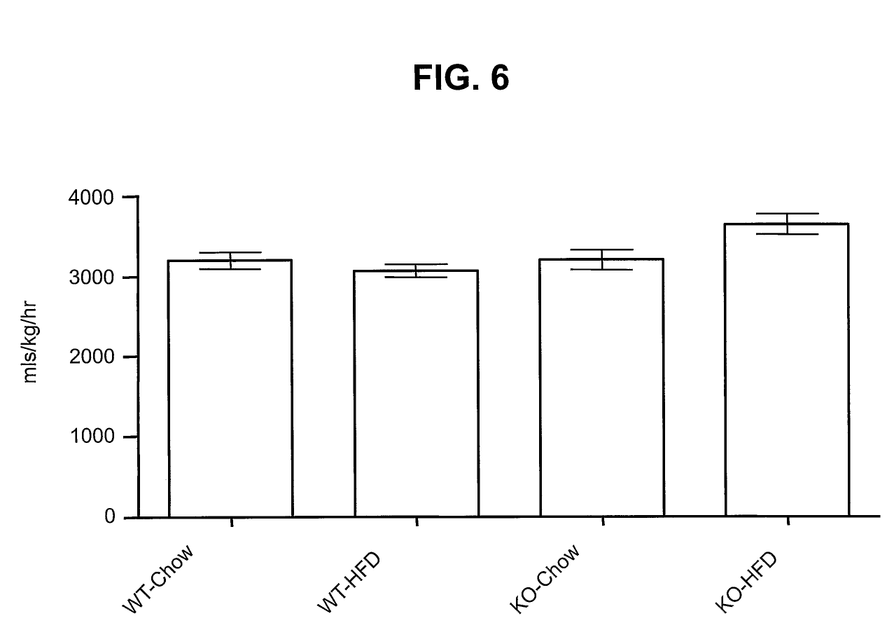

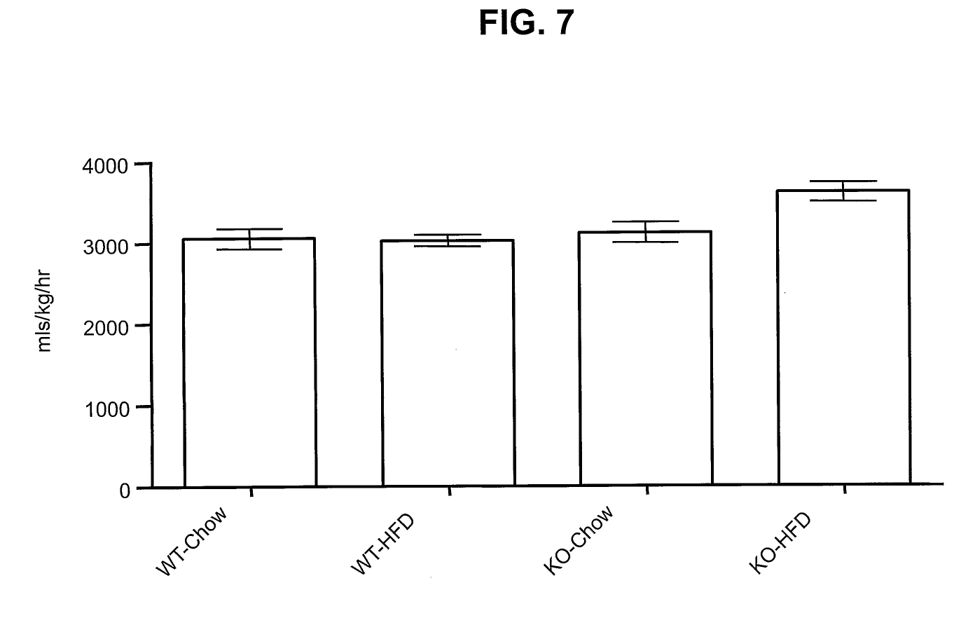

Changes in metabolic rate were evident, however, in the HFD-fed PDE10 KO mice. Oxygen consumption (VO2), measured as either total VO2 (FIG. 6) or resting VO2 (FIG. 7), was increased in the HFD-fed KO mice. Resting VO2 was elevated 19% over the values for the chow-fed PDE10 KO mice. The resting VO2 values for the wild type mice on both the HFD and chow diet were equivalent to the chow-fed PDE10 KO mice.

### Example 2

### Effect of PDE10 Inhibition on Metabolic Parameters in Mice Fed a High Fat Diet

### KO Mice

Table 1 below compares plasma metabolites (mean±S.E.M.) for each of the chow-fed and HFD-fed wild-type and PDE10 KO groups. While on a chow diet, the PDE10 KO mice exhibited reduced fat pad weight, triglycerides, FFA, and leptin, as well as a trend towards reduced cholesterol and hemoglobin A1c (HbA1c), as compared to chow-fed wild type mice. While adiponectin showed a trend towards reduction in chow-fed PDE10 KO mice, when normalized for grams of fat, adiponectin was increased in the PDE10 KO mice. When fed a HFD, the PDE10 KO mice, as compared to wild type, demonstrated a reduction in fat pad weight, triglycerides, FFA, leptin, and insulin. The HFD-fed PDE10 KO mice also demonstrated a trend towards reduced cholesterol, adiponectin, and fructosamine. As with the chow-fed mice, the HFD-fed PDE10 KO mice also demonstrated an increase in adiponectin when the value was normalized for grams of fat.

TABLE 1
Plasma Metabolites Following High-fat Diet

| | Chow Diet | | | HFD | | |
|---|---|---|---|---|---|---|
| | WT | KO | p | WT | KO | p |
| Body Temp. | 98.5 ± 0.4 | 98.3 ± 0.3 | N.S. | 98.7 ± 0.1 | 98.3 ± 0.3 | N.S. |
| ° F. | | | | | | |
| Fat Pad Wt. | 0.4 ± 0.1 | 0.2 ± 0.02 | 0.008 | 1.6 ± 0.1 | 0.4 ± 0.1 | >0.001 |
| G | | | | | | |
| cGMP | 7.5 ± 1.5 | 8.1 ± 1.2 | N.S. | 6.6 ± 0.3 | 8.9 ± 0.7 | N.S. |
| pmol/ml | | | | | | |
| Glucose | 173.1 ± 7.2 | 159.8 ± 14.5 | N.S. | 192.0 ± 19.1 | 200.5 ± 4.8 | N.S. |
| mg % | | | | | | |
| Insulin | 2.0 ± 0.3 | 1.7 ± −.2 | N.S. | 4.6 ± 0.6 | 2.8 ± 0.3 | 0.006 |
| ng/ml | | | | | | |
| Triglycerides | 215.8 ± 21.5 | 124.2 ± 13.4 | 0.002 | 372.2 ± 68.5 | 191.9 ± 19.9 | 0.010 |
| mg % | | | | | | |
| Cholesterol | 253.0 ± 23.2 | 175.3 ± 31.0 | 0.060 | 280.4 ± 54.3 | 214.3 ± 13.7 | 0.207 |
| mg/dl | | | | | | |
| FFA | 520.7 ± 66.6 | 274.0 ± 34.0 | 0.004 | 688.5 ± 109.9 | 389.1 ± 41.9 | 0.011 |
| μEq/l | | | | | | |
| Fructosamine | 196.1 ± 1.9 | 197.3 ± 2.0 | N.S. | 192.0 ± 3.9 | 185.6 ± 2.2 | 0.145 |
| μmol/l | | | | | | |
| HbA1c | 4.9 ± 0.04 | 4.8 ± 0.1 | 0.11 | 5.0 ± 0.0 | 4.8 ± 0.1 | 0.029 |
| % | | | | | | |
| Adiponectin | 8.6 ± 0.4 | 7.9 ± 0.2 | 0.12 | 8.2 ± 0.3 | 7.6 ± 0.1 | 0.056 |
| μg/ml | | | | | | |
| Adiponectin | 27.2 ± 4.2 | 40.9 ± 4.3 | 0.04 | 5.1 ± 0.3 | 26.7 ± 3.3 | >0.001 |
| μg/ml/g fat | | | | | | |
| Leptin | 1255 ± 292 | 608 ± 106 | 0.05 | 10488 ± 1914 | 2151 ± 380 | >0.001 |
| pmol/ml | | | | | | |

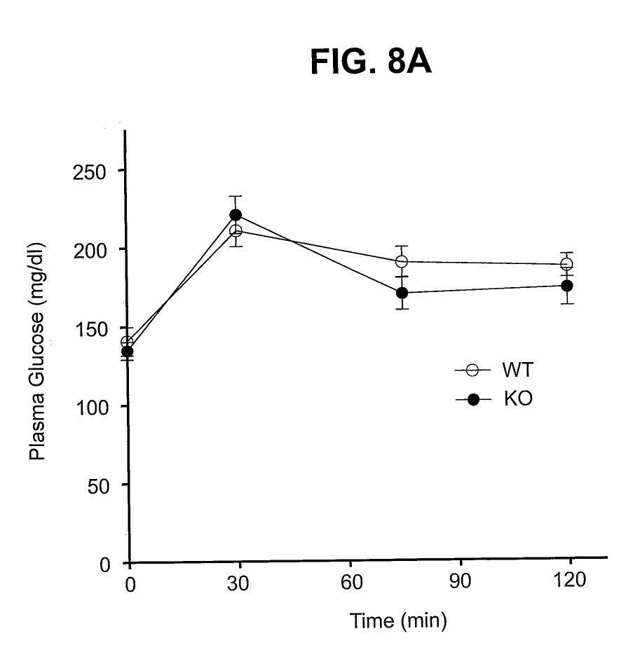

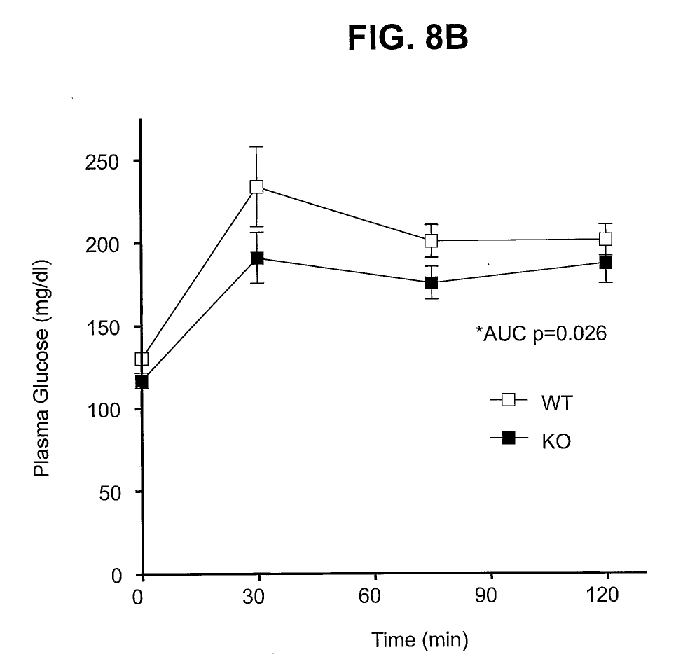

In addition to PDE10 KO mice being relatively resistant to developing hyperinsulinemia while fed a HFD, the PDE10 KO mice were also relatively resistant to developing glucose intolerance. As shown in FIG. 8B, the area under the curve for the HFD-fed wild type mice was greater than for the HFD-fed PDE10 KO mice. This difference in glucose tolerance was not evident when comparing chow-fed wild type and PDE10 KO mice (see FIG. 8A).

### Example 3

### The Effect of PDE10 Inhibitor Administration on Fasting-Induced Refeeding

### CD Rats

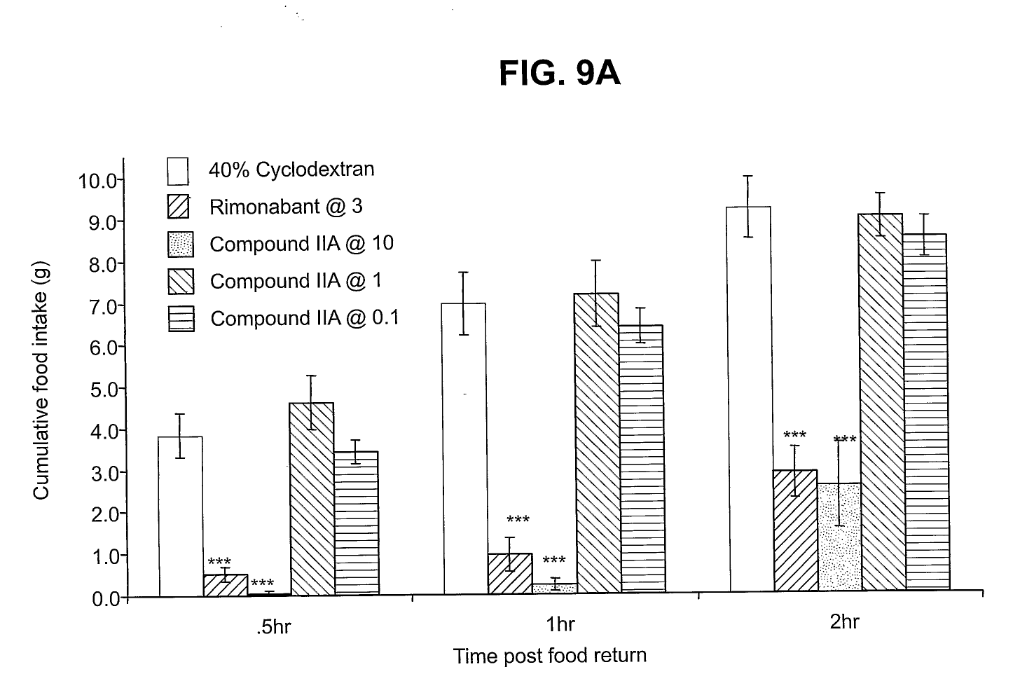

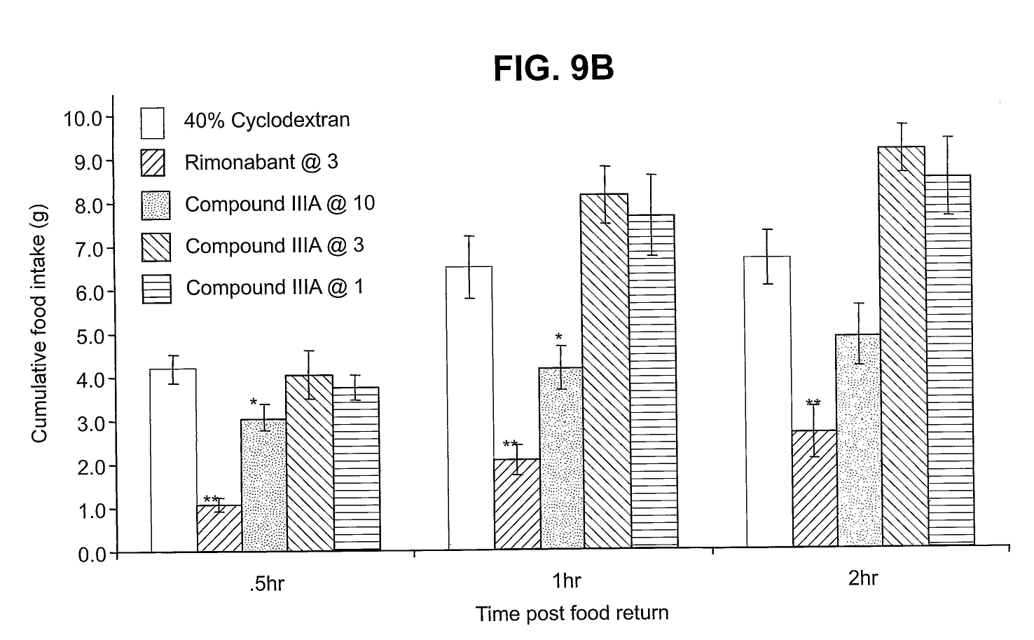

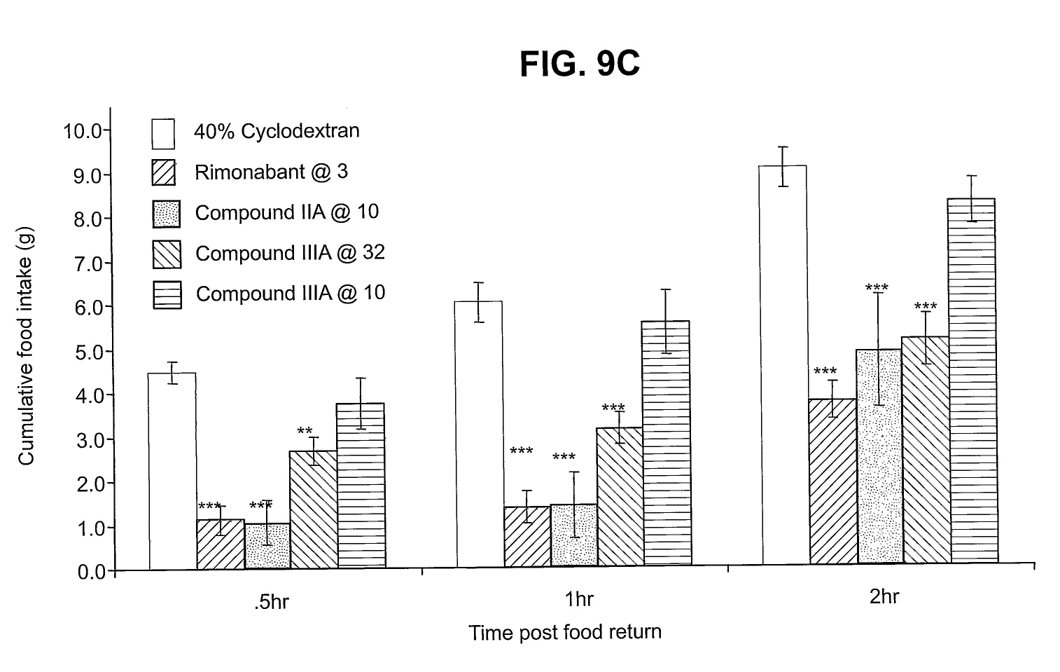

Compound IIA, Compound IIIA and rimonabant were tested in the fasted-induced feeding assay described above. Compound IIA was tested at 10 mg/kg, 1 mg/kg and 0.1 mg/kg; Compound IIIA was tested at 32 mg/kg, 10 mg/kg, 3 mg/kg and 1 mg/kg; and rimonabant was tested at 3 mg/kg. Both Compound IIA and Compound IIIA were efficacious at 10 mg/kg. Both Compound IIA and Compound IIIA were shown to reduce fasting-induced refeeding in the rats in a dose dependent fashion as compared to a control compound (CB-1 antagonist—Rimonabant) which is known to promote weight loss in humans (See, FIGS. 9A, 9B and 9C).

### Example 4

### The Effect of PDE10 Inhibitor Administration on Body Weight

### DIO Mice

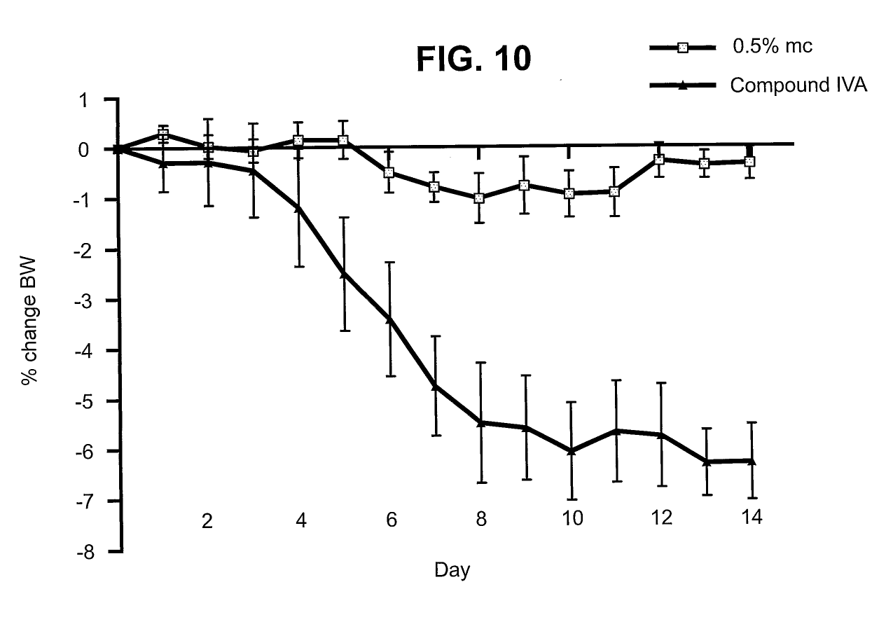

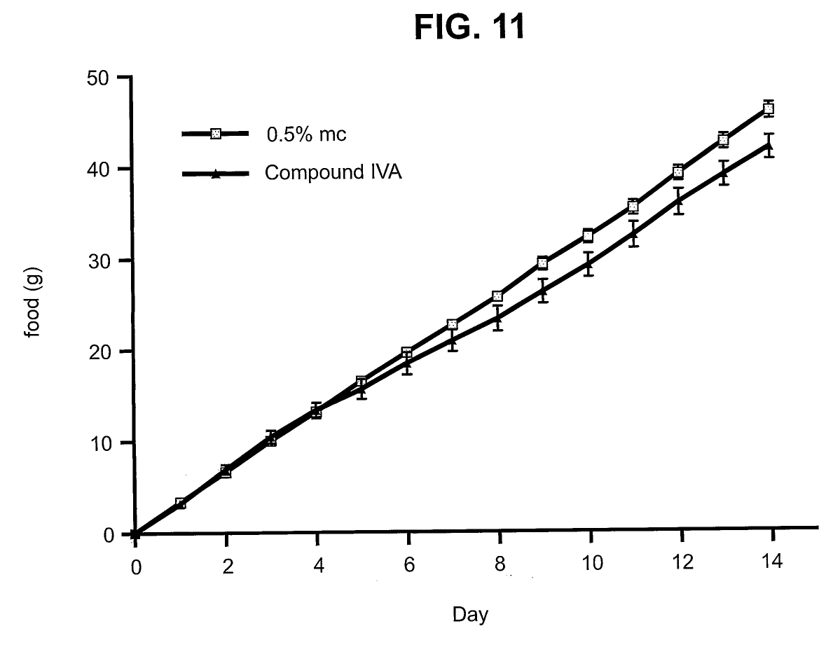

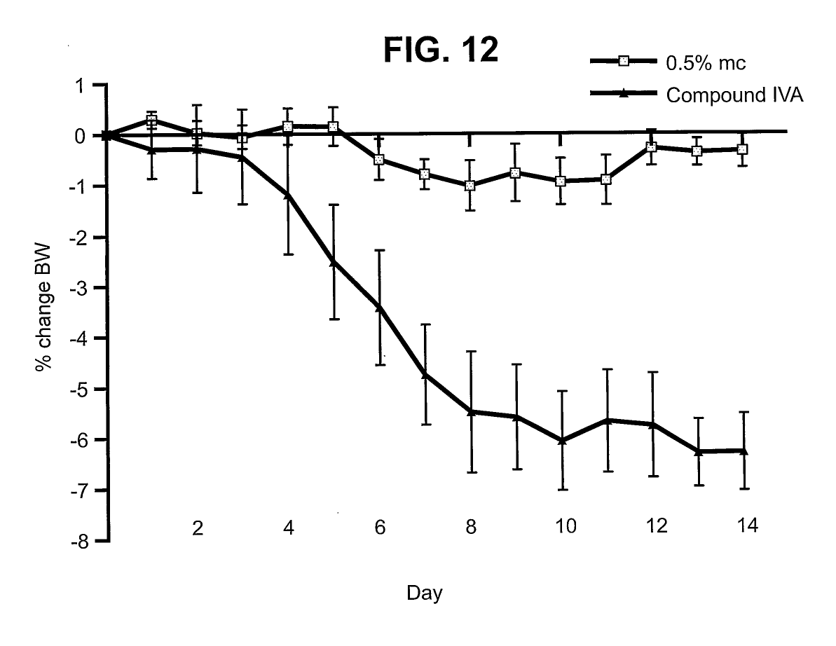

DIO mice were treated with Compound IVA at a dose of 15 mg/kg administered twice daily (po). Control mice were administered the vehicle, 0.5% methylcellulose. Treatment with Compound IVA led to weight loss in the DIO mice (FIG. 10). The onset of weight loss was after three days treatment (apparent on the 4^{th} day). There was also a small reduction in food intake in the Compound IVA treated mice which contributed to the weight loss (see, FIG. 11). Oxygen consumption was determined in the Compound IV and vehicle treated mice at the end of the study. There was a slight non-significant increase in oxygen consumption in the Compound IV treated mice (see, FIG. 12). These result indicate that a chronic treatment with a PDE10A inhibitor may have salutary effects against obesity.

The results of the Examples described above demonstrate that PDE10 inhibition is an effective means of reducing body weight, reducing body fat, and treating disorders associated with increased adiposity. These results also demonstrate that PDE10 inhibitors are also effective in treating disorders associated with NIDDM, glucose intolerance, insulin resistance and metabolic syndrome in addition to reducing body weight, body fat, and treating disorders associated with increased adiposity.

1. A method of treating a subject to reduce body fat or body weight, or to treat non-insulin dependent diabetes, metabolic syndrome, or glucose intolerance, comprising administering to a subject in need thereof a therapeutically effective amount of a phosphodiesterase 10 (PDE10) antagonist.

2. The method of claim 1, wherein the subject is overweight.

3. The method of claim 2, wherein the subject is obese.

4. The method of claim 1, wherein the PDE10 antagonist is a PDE10 selective antagonist.

5. The method of claim 1, wherein the PDE10 antagonist is orally administered to the subject.

6. The method of claim 1, wherein the subject is a human.

7. The method of claim 1, wherein the PDE10 antagonist is PDE10 selective, the PDE10 antagonist is orally administered, and the subject is human.

8. The method of claim 1, further comprising administering a second therapeutic agent to the subject.

9. The method of claim 8, wherein the second therapeutic agent is an anti-obesity agent.

10. The method of claim 9, wherein the anti-obesity agent is selected from the group consisting of rimonabant, orlistat, sibutramine, bromocriptine, ephedrine, leptin, pseudoephedrine, peptide YY₃₋₃₆, and analogs thereof.

11. (canceled)

12. A method for identifying an agent that can be used to reduce body fat or body weight, or to treat NIDDM, metabolic syndrome, or glucose intolerance, comprising (i) administering a candidate PDE10 antagonist to a test subject, and (ii) determining whether the PDE10 antagonist is effective in reducing body fat or body weight, or in treating a symptom of NIDDM, metabolic syndrome, or glucose intolerance in the test subject.

13. The method of claim 12, further comprising testing the candidate PDE10 antagonist in an in vitro test for PDE10 antagonist activity prior to administering the candidate PDE10 antagonist to the test subject.

14. A kit comprising an PDE10 antagonist and instructions for administering the antagonist to a subject to reduce the body fat, body weight, or to treat non-insulin dependent diabetes (NIDDM), metabolic syndrome, or glucose intolerance, in the subject.

15. The kit of claim 14, wherein the PDE10 antagonist is a PDE10 selective antagonist.

16. (canceled)

17. The kit of claim 14, further comprising a second therapeutic agent.

18. The kit of claim 17, wherein the second therapeutic agent is an anti-obesity agent.

19. The kit of claim 18, wherein the anti-obesity agent is selected from the group consisting of rimonabant, orlistat, sibutramine, bromocriptine, ephedrine, leptin, pseudoephedrine, peptide YY₃₋₃₆, and analogs thereof.
